(19) United States
(12) Patent Application Publication
(10) Pub. No.: US 2026/0160757 A1
(43) Pub. Date: Jun. 11, 2026

(54) Chondroitin Sulfate and Heparan Sulfate as Biomarkers for Discriminating Osteoarthritis and Rheumatoid Arthritis

(71) Applicant: Elypta AB, Solna (SE)

(72) Inventors: Francesco Gatto, Stockholm (SE); Fredrik Härenstam, Stockholm (SE)

(21) Appl. No.: 19/126,629

(22) PCT Filed: Nov. 2, 2023
(86) PCT No.: PCT/EP2023/080602

§ 371 Date: May 1, 2025

(30) Foreign Application Priority Data
Nov. 2, 2022 (GB) 2216295.2

(51) Int. Cl. G01N 33/53

(52) U.S. Cl. CPC G01N 33/5308; G01N 2400/40; G01N 2800/102; G01N 2800/105

(57) ABSTRACT

The present invention relates to methods of screening for osteoarthritis (OA) and rheumatoid arthritis (RA) and methods of discriminating between OA and rheumatoid arthritis RA in a subject having arthritis or in a subject suspected of having arthritis, said methods comprising determining the level in a body fluid sample of one or more glycosaminoglycan (GAG) property in accordance with the invention. The present invention also provides methods for monitoring the progression of OA or RA in a subject having arthritis.

The present invention relates to biomarkers associated with osteoarthritis (OA) and rheumatoid arthritis (RA), and to methods of screening for osteoarthritis or rheumatoid arthritis. Such methods involve determining the level and/or composition of certain biomarkers which are indicative of OA or RA.

Arthritis is a common, and often debilitating, group of conditions that affects the health of joints in the body. Arthritis can affect people of all ages, including children. Arthritis includes osteoarthritis (OA) and rheumatoid arthritis (RA), i.e. OA and RA are different types of arthritis.

OA is characterised by the deterioration of cartilage that cushions the ends of bones in joints. OA is sometimes referred to as “wear and tear” arthritis. OA is a condition that typically results in painful and stiff joints, and swelling. In the UK, OA is the most common type of arthritis. OA is a commonly misdiagnosed condition and is often only diagnosed very late, after the disease has progressed and cartilage damage has become irreversible.

RA is an autoimmune disease. In RA, the immune system attacks cells that line joints. Like OA, RA is also a condition that typically results in painful and stiff joints, and swelling.

The treatments, or other clinical management strategies, for RA and OA are typically different (i.e. different for RA versus OA). By way of an example, subjects having RA may be prescribed disease-modifying anti-rheumatic drugs (DMARDs). DMARDS are not conventionally prescribed for OA as no clinically significant pain relief is offered by DMARDs in OA.

As is evident from the above discussion, although OA and RA are both types of arthritis and are both typically characterized by having common symptoms (e.g. joint pain, stiffness and swelling), OA and RA are distinct conditions which have distinct underlying causes and which are treated differently in the clinic (e.g. with different treatments or other clinical management strategies).

As OA and RA have symptoms in common with each other, it can be difficult for clinicians, with existing screening or diagnostic methods (e.g. radiological imaging methods) to determine whether arthritis patients, or suspected arthritis patients, have OA or RA, i.e. it can be difficult to discriminate between OA and RA.

There is an urgent need to improve the current screening and diagnostics landscape for arthritis. In particular, affordable and practical tools for arthritis diagnostics are needed to assist healthcare professionals, for example to assist in determining whether subjects have osteoarthritis (OA) or rheumatoid arthritis (RA). This would help to guide the clinical management of arthritis patients.

Circulating biomarkers are molecules that can be measured in accessible body fluids of individuals, e.g. urine, and whose levels are useful to assist in, for example, screening for disease, diagnosis of disease, and monitoring of disease progression.

What is needed in the art are new methods of screening for OA and RA (e.g. for distinguishing between OA and RA). The identification of novel biomarkers for OA and RA may potentially have clinical implications for a large number of patients and would be an important clinical advancement. Here, the inventors provide methods that employ certain markers which, advantageously, are useful in screening for OA and RA (e.g. to distinguish between OA and RA). Advantageously, such methods are non-invasive and can be performed on readily obtainable samples.

Surprisingly and advantageously, the present inventors have identified that the level of certain glycosaminoglycans (GAGs) and/or the chemical compositions of said GAGs, are different in body fluid samples from OA patients in comparison to RA patients. These differential levels of the GAGs CS or HS, or differential chemical compositions of the GAGs CS or HS (GAG profiles) as characterized by differential levels of certain GAG properties in accordance with the present invention, can act as biomarkers useful in screening for OA and RA (e.g. to distinguish between OA and RA). Clearly the finding that OA and RA screening (e.g. discrimination between OA and RA) can be carried out using an accessible body fluid sample, e.g. urine, from a subject is extremely advantageous.

Thus, in one aspect the present invention provides a method of screening for osteoarthritis (OA) or rheumatoid arthritis (RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of:
(i) 4S CS,
(ii) NS HS,
(iii) 0S HS,
(iv) total concentration of CS; and
(v) total concentration of HS,
wherein said sample has been obtained from said subject.

In some embodiments, methods of the present invention comprise determining the level of 4S CS.

In some embodiments, methods of the present invention comprise determining the level of NS HS.

In some embodiments, methods of the present invention comprise determining the level of 0S HS.

In some embodiments, methods of the present invention comprise determining the total concentration of CS.

In some embodiments, methods of the present invention comprise determining the total concentration of HS.

In some embodiments, methods of the present invention comprise determining the level of 4S CS or NS HS.

In some embodiments, methods of the present invention comprise determining the level of 4S CS and NS HS.

In some embodiments, methods of the present invention comprise determining the level of one or more GAG property selected from the group consisting of:
(i) absolute concentration of 4S CS,
(ii) absolute concentration of NS HS and/or relative concentration of Ns HS,
(iii) absolute concentration of 0S HS,
(iv) total concentration of CS; and
(v) total concentration of HS.

The terms “absolute concentration”, “relative concentration” and “total concentration” are discussed elsewhere herein.

Unless otherwise clear from the context, the term “one or more” includes “all”.

In some embodiments, methods of the present invention comprise determining the absolute concentration of 4S CS.

In some embodiments, methods of the present invention comprise determining the absolute concentration of NS HS.

In some embodiments, methods of the present invention comprise determining the relative concentration of NS HS.

In some embodiments, methods of the present invention comprise determining the absolute concentration of 0S HS.

In some embodiments, methods of the present invention comprise determining the total concentration of CS.

In some embodiments, methods of the present invention comprise determining the total concentration of HS.

In some embodiments, methods of the present invention comprise determining the absolute concentration of 4S CS or the absolute concentration of NS HS.

In some embodiments, methods of the present invention comprise determining the absolute concentration of 4S CS and NS HS.

Thus, in preferred embodiments of the present invention, the level of 4S CS is the absolute concentration of 4S CS, the level of NS HS is the absolute concentration of NS HS or the relative concentration of NS HS (more preferably the absolute concentration of NS HS) and the level of 0S HS is the absolute concentration of 0S HS.

Unless otherwise clear from the context, references to one or more GAG properties (or GAG forms or GAG features or GAGome features or GAGome properties) “in accordance with the invention” or “in accordance with the present invention” (or equivalent phrases) are references to 4S CS (preferably absolute concentration of 4S CS), NS HS (preferably absolute concentration of NS HS and/or relative concentration of NS HS, more preferably absolute concentration of NS HS), 0S HS (preferably absolute concentration of 0S HS), total concentration of CS and/or total concentration of HS. Particularly preferred GAG properties “in accordance with the invention” or “in accordance with the present invention” are 4S CS (preferably absolute concentration of 4S CS) and/or NS HS (preferably absolute concentration of NS HS).

In preferred methods of the invention, an altered level (increased or decreased as the case may be) of one or more GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) in comparison to a control level is indicative of either OA or RA in said subject (indicative of whether said subject has either OA or RA).

In certain methods of the invention, an altered level (e.g. increased or higher level) of one or more GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) in comparison to a control level is indicative of OA in said subject (indicative of whether said subject has OA).

In certain methods of the invention, an altered level (e.g. decreased or lower level) of one or more GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) in comparison to a control level is indicative of RA in said subject (indicative of whether said subject has RA).

The skilled person would be readily able to establish appropriate control levels for use in accordance with the invention. Certain appropriate control levels and how an indication of OA or RA can be reached (discrimination of likely OA versus likely RA) are discussed elsewhere herein.

In some embodiments of methods of the invention, a level of one or more of the GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) that lies within an established (or given or appropriate) reference range (or reference interval) or a level that is altered (increased or decreased as the case may be) with respect to an established (or given or appropriate) reference range (or reference interval) is indicative of either OA or RA in said subject (indicative of whether said subject has either OA or RA).

The skilled person would be readily able to establish appropriate reference ranges (or reference intervals) for use in accordance with the invention. Certain appropriate reference ranges (or reference intervals) and how an indication of OA or RA can be reached using such reference ranges are discussed elsewhere herein.

In preferred methods of the invention both the level and the chemical composition are determined. In other preferred methods of the invention the chemical composition alone is determined, or, in other preferred methods, the level (total level or total concentration) of CS and/or HS alone is determined.

Methods of the present invention comprise determining the level and/or chemical composition of one or both of the glycosaminoglycans (GAGs) chondroitin sulfate (CS) and heparan sulfate (HS) in a body fluid sample. In some embodiments, the level and/or chemical composition of one of said GAGs is determined. In some embodiments, the level and/or chemical composition of chondroitin sulfate (CS) is determined. In some embodiments, the level and/or chemical composition of heparan sulfate (HS) is determined. In some embodiments, the level and/or chemical composition of chondroitin sulfate (CS) and heparan sulfate (HS) is determined.

As described elsewhere herein, in some preferred embodiments of methods of the present invention, the method comprises determining the level and/or chemical composition of the protein-free fraction of one or both of the glycosaminoglycans (GAGs) chondroitin sulfate (CS) and heparan sulfate (HS) in a body fluid sample. Thus, in some embodiments, the level and/or chemical composition of the protein-free fraction of chondroitin sulfate (CS) is determined. In some embodiments, the level and/or chemical composition of the protein-free fraction of heparan sulfate (HS) is determined. In some embodiments, the level and/or chemical composition of the protein-free fraction of chondroitin sulfate (CS) and heparan sulfate (HS) is determined.

Although in some embodiments of methods of the invention the method comprises determining the level and/or chemical composition of the protein-free fraction of one or both of the glycosaminoglycans (GAGs) chondroitin sulfate (CS) and heparan sulfate (HS), it is not essential in all aspects of the invention for the level and/or chemical composition specifically of the “protein-free fraction” to be determined.

Glycosaminoglycans (GAGs) are sugar containing molecules which can be attached to proteins on serine residues, i.e. can form a part of a proteoglycan. They are formed from linear or unbranched chains of monosaccharides (i.e. are polysaccharides) which can be sulfated. Heparan sulfate (HS), chondroitin sulfate (CS), keratan sulfate (KS), hyaluronic acid (HA) and heparin are the common types of GAG, of which HS and CS are examples of sulfated GAGs. The different types of GAG are distinguished by different repeating disaccharide units.

When linked or attached to proteins, CS and HS are GAGs that share a common biosynthetic route in the linkage to the core protein, but thereafter they differ in their polymerisation in that the CS repeating disaccharide is made up of repeating N-acetylgalactosamine (GalNAc) and glucuronic acid residues (GlcA), whilst the repeating disaccharide in HS is typically made up of repeating N-acetylglucosamine (GlcNAc) and glucuronic acid (GlcA) residues. Each monosaccharide is attached by a specific enzyme allowing for multiple levels of regulation over GAG synthesis.

Although GAGs can be attached to proteins, i.e. they may be in a protein-bound or proteoglycan form, GAGs can also exist in a “free” form, i.e. they can also exist in a non protein-bound or non-proteoglycan form. Such “free” forms of GAGs are referred to herein as “protein-free GAGs”.

Thus, in body fluids (or body samples) there is typically a protein-free fraction of GAGs (or protein-free pool of GAGs) and a protein-bound fraction of GAGs (or protein-bound pool of GAGs). Together (i.e. protein-free fraction plus protein-bound fraction), the two fractions may be referred to as the entire GAG fraction or entire GAG pool.

In preferred methods of the invention, the level or composition of the protein-free fraction of one or both of the GAGs chondroitin sulfate (CS) and heparan sulfate (HS) in a body fluid sample is determined.

The protein-free fraction GAGs (or disaccharide units derived therefrom as discussed elsewhere herein) for analysis may be obtained by any suitable means.

As discussed elsewhere herein, body fluid samples are typically processed prior to analysis. Such processing typically comprises subjecting the GAGs to a processing step to obtain disaccharide units for analysis. Such a processing step typically comprises contacting said sample (or said GAGs in said sample) with an enzyme (e.g. a GAG lyase such as a chondroitinase or a heparinase) which digests (or fragments) the GAGs into disaccharide units. Without wishing to be bound by theory, such enzymes are not able to access protein-bound GAGs but rather act on (or use as their substrate) only (or essentially only) protein-free GAGs. If proteoglycans (which are proteins with GAGs bound or attached thereto) are contacted with a proteolytic agent (e.g. a protease such as proteinase K) the protein component thereof is digested by the proteolytic agent and the protein-bound GAGs are freed or released, meaning that protein-bound GAGs are (or are converted into) into a “free” or “released” form, which would then be available for digestion (or fragmentation) by an enzyme such as a GAG lyase. As in certain preferred methods of the invention it is specifically the level and/or composition of the protein-free fraction (or naturally protein-free fraction) of one or both of the GAGs CS and HS that is determined, then preferred methods of the invention do not comprise contacting the sample with a proteolytic agent (e.g. a protease such as proteinase K). Omitting a proteolytic agent during processing of a sample for analysis is thus a way to obtain (or obtain only or obtain essentially only) the protein-free fraction of GAGs (or disaccharides subsequently derived therefrom) for analysis. Protein-bound GAGs (proteoglycan GAGs) that have been (or become) “freed” or “released” by the action of a proteolytic agent (e.g. a protease) are not protein-free GAGs in accordance with the present invention.

Thus, protein-free GAGs (or the protein-free fraction of GAGs) are GAGs (or the fraction of GAGs) that are already (or naturally) free (i.e. not protein-bound) in the absence of (or without) the sample having been treated with a proteolytic agent (e.g. a protease). Put another way, the protein-free GAGs (or the protein-free fraction of GAGs) are GAGs (or the fraction of GAGs) that are free (i.e. not protein-bound) in an original, or initial, or unprocessed sample. For example, the protein-free GAGs (or the protein-free fraction of GAGs) can be GAGs (or the fraction of GAGs) that are present in a sample, e.g. an original or unprocessed sample, and are susceptible to, or accessible to, or available for (e.g. are a substrate for) digestion (or fragmentation) into disaccharide units as described elsewhere herein, e.g. using an enzyme such as a lyase enzyme.

As indicated above, protein-free GAGs (or the protein-free fraction of GAGs) may be considered the non-protein bound (or non-protein bound fraction) or non-proteoglycan form (or non-proteoglycan fraction) of GAGs. Put another way, the protein-free GAGs (or the protein-free fraction of GAGs) are GAGs not decorating a proteoglycan in the original, or initial, or unprocessed sample.

Thus, in some preferred embodiments, methods comprise determining the level and/or chemical composition of the protein-free fraction of one or both of the glycosaminoglycans (GAGs) chondroitin sulfate (CS) and heparan sulfate (HS) in accordance with the invention, in a body fluid sample, wherein the sample is subjected to processing prior to determining said level and/or composition and wherein said processing does not comprise contacting said sample with a proteolytic agent (e.g. a protease such as proteinase K), or other agent which can release protein-free GAGs from protein-bound GAGs.

Alternatively viewed, in some preferred embodiments, methods comprise determining the level and/or chemical composition of the protein-free fraction of one or both of the glycosaminoglycans (GAGs) chondroitin sulfate (CS) and heparan sulfate (HS) in accordance with the invention in a body fluid sample, wherein the sample is subjected to processing prior to determining said level and/or composition and wherein said processing does not comprise contacting said sample with a proteolytic agent (e.g. a protease such as proteinase K), or other agent which can release GAGs (or GAG chains) from proteoglycans.

Thus, in some preferred embodiments, methods comprise determining the level and/or chemical composition of the protein-free fraction of one or both of the glycosaminoglycans (GAGs) chondroitin sulfate (CS) and heparan sulfate (HS) in accordance with the invention in a body fluid sample, wherein said sample has been obtained from said subject and has been subjected to processing prior to determining said level and/or chemical composition, wherein said processing
(a) comprises fragmenting said one or both GAGs into disaccharide units (e.g. as described elsewhere herein); and
(b) does not comprise prior to (a) contacting said sample with an agent which can release GAGs (or GAG chains) from proteoglycans.

Thus, in some preferred embodiments, methods comprise determining the level and/or chemical composition of the protein-free fraction of one or both of the glycosaminoglycans (GAGs) chondroitin sulfate (CS) and heparan sulfate (HS) in accordance with the invention in a body fluid sample, wherein said sample has been obtained from said subject and has been subjected to processing prior to determining said level and/or chemical composition, wherein said processing
(a) comprises fragmenting said one or both GAGs into disaccharide units (e.g. as described elsewhere herein); and
(b) does not comprise prior to (a) contacting said sample with a proteolytic agent.

As indicated above, protein-bound GAGs (or the protein-bound fraction of GAGs) may be considered proteoglycan GAGs (or the proteoglycan fraction of GAGs). Alternatively viewed, protein-bound GAGs (or the protein-bound fraction of GAGs) may be considered as GAGs that typically require the protein to which they are bound to be contacted with a proteolytic agent (e.g. a protease such as a non-specific protease) in order for them to be (or become) freed or released.

In some other embodiments of methods of the invention the level and/or chemical composition in accordance with the invention of the entire fraction (or entire pool) of one or both of the GAGs CS and HS in a body fluid sample may be determined (i.e. protein-free GAGs plus protein-bound GAGs). In such embodiments, the sample is typically contacted with a proteolytic agent during processing of the sample.

The “level” of HS or CS as referred to herein generally refers to the total level or amount (e.g. concentration) of the HS or CS present in the sample. The level of CS and/or HS in a sample can be measured or determined by any appropriate method which would be well-known and described in the art. Some methods involve electrophoresis, in particular capillary electrophoresis, e.g. capillary electrophoresis with fluorescence detection, e.g. laser-induced fluorescence detection. Other suitable methods are gel electrophoresis, e.g. agarose gel electrophoresis (e.g. FACE, fluorophore-assisted carbohydrate electrophoresis) or mass spectrometry or liquid chromatography, e.g. HPLC, optionally in combination with mass spectrometry (HPLC-MS). Preferred methods involve high performance liquid chromatography (HPLC), preferably ultra-HPLC (UHPLC), in combination with mass spectrometry, e.g. MS/MS or triple quadrupole mass spectrometry. Preferred methods comprise ultra-high-performance liquid chromatography (UHPLC) coupled with electrospray ionization triple-quadrupole mass spectrometry system.

Conveniently these levels can be measured as a concentration (e.g. a real or absolute level or concentration), for example, as a number of microgram per ml (μg/ml). However, again, any appropriate measure of level may be used.

In typical methods of the present invention the levels of HS and/or CS (e.g. the levels of the protein-free fraction of HS and/or CS) are determined separately or individually. In other words the methods do not involve the measurement of total GAG levels in a sample or the total levels of all the GAGs present in combination (e.g. in the protein-free GAG fraction) but involve the measurement of the levels of one or more of the individual GAGs HS or CS.

In particular embodiments, the level (e.g. total level, or concentration) of CS and/or HS (e.g. the level of the protein-free fraction of CS and/or HS), can be determined in body fluid samples, for example in urine samples.

The individual monosaccharide units making up the CS and HS can have different sulfation patterns in terms of the position of the sulfate molecules and the amount/number of sulfate molecules. For CS, sulfation may most commonly occur at one or more of position 2 of the GlcA and positions 4 and 6 of the GalNAc. For HS, sulfation may occur at one or more of position 2 of the GlcA after epimerization to IdoA (iduronic acid), positions 3 and 6 of the GlcNAc, and N-sulfation of the GlcNAc. Thus, each individual disaccharide in the GAG chain may have 0 (i.e. be unsulfated), 1, 2, 3 or 4 (only in HS) sulfation forms and this in turn gives rise to different overall chemical compositions of GAG chains in terms of sulfation levels and specific disaccharide sulfation patterns.

As described elsewhere herein, preferred embodiments of the invention involve the determination of the chemical composition of one or both of CS and HS. The term “chemical composition” as used herein can refer to both the levels of the GAGs as well as the disaccharide sulfation composition of the GAGs. In particular, this term includes a determination of one or more particular forms, e.g. sulfation forms, of the disaccharides making up the CS or HS GAGs. Put another way, the term “chemical composition” refers to the amount or level of one or more of the various sulfated and/or unsulfated forms of CS or HS disaccharides, as well as, for example, some other properties of the individual GAGs present, such as total HS or CS GAG levels, or other properties related to GAG sulfation such as HS charge or CS charge as described further elsewhere herein. Such a chemical composition which is analysed or determined in the present invention can also be referred to herein as a GAG profile, GAG forms, GAG features, GAG properties, GAGome, GAGome features.

Thus, for example, the term “chemical composition” as used herein may refer to a determination or analysis of the sulfation patterns (e.g. one or more of the sulfation forms) of the disaccharides making up CS and/or HS.

As is evident from the above discussion, in accordance with the present invention it is essential that the level of one or more GAG property selected from the group consisting of 4S CS (preferably the absolute concentration of 4S CS), NS HS (preferably the absolute concentration of NS HS), 0S HS (preferably the absolute concentration of 0S HS), total concentration of CS and total concentration of HS is determined. Thus, in accordance with the present invention and disclosure, determining the “chemical composition” must include a determination of the level of one or more GAG property selected from the group consisting of 4S CS, NS HS, 0S HS, total concentration of CS and total concentration of HS. The level of one or more other (additional) GAG properties (e.g. as described herein) may additionally be determined (or measured), but in accordance with the present invention and disclosure it is the level of one or more GAG property selected from the group consisting of 4S CS, NS HS, 0S HS, total concentration of CS and total concentration of HS that allows an indication (e.g. diagnosis) of OA or RA to be made (or reached).

For CS, there are 8 main sulfated and unsulfated forms (sulfation patterns, disaccharide sulfation forms) which are: 0S CS (also referred to as unsulfated CS or CS O unit), 2S CS (also referred to as chondroitin-2-sulfate), 4S CS (also referred to as chondroitin-4-sulfate or CS A unit), 6S CS (also referred to as chondroitin-6-sulfate or CS C unit), 2S4S CS (also referred to as chondroitin-2-4-sulfate), 2S6S CS (also referred to as chondroitin-2-6-sulfate or CS D unit), 4S6S CS (also referred to as chondroitin-4-6-sulfate or CS E unit) and Tris CS (also referred to as chondroitin-2-4-6-sulfate or trisulfated CS).

Each of the above is a form of CS GAG (a CS GAG form or property or feature or GAGome feature) which may be measured in the methods of the present invention. One or more of these forms may be measured, for example up to 8, e.g. 1, 2, 3, 4, 5, 6, 7 or all 8 of these sulfation forms may be measured. In some embodiments, measurement of all 8 of these sulfation forms is preferred. In some embodiments, measurement of, or of at least, the CS sulfation forms 0S CS, 6S CS, 4S CS, 2S6S CS, 2S4S CS, 4S6S CS is preferred.

Another GAG property for CS which may be measured in the methods of the present invention is the total concentration of CS (which may also be referred to as CS tot or Tot CS or total CS) or the total level of CS. This is typically measured as a concentration, e.g. an absolute concentration, e.g. in μg/ml, as described elsewhere herein. In some embodiments, the total CS is measured by summing the level of all measured CS disaccharide forms listed above (the 8 main sulfated and unsulfated forms). Thus, total CS may be the sum of the levels of all measured CS disaccharide forms listed above (the 8 main sulfated and unsulfated forms). In some embodiments, the total CS is measured by summing the level of all of the CS disaccharide forms that are measured at (or above) an analytically detectable level, or at (or above) a level that has been selected as a minimum threshold level (a minimum threshold level that is considered analytically detectable or analytically meaningful, for example, the limit of detection or the lower limit of quantification for such CS disaccharide form). For example, the total CS may be the sum of all of the CS disaccharide forms that are measured at a level at, or above, a given analytically detectable level, or at, or above, a selected minimum threshold level (e.g. at a concentration of >0.1 μg/ml). The level of CS disaccharide forms below a given analytically detectable level or below a minimum threshold level would typically provide only a negligible (or de minimis) contribution to the overall CS concentration, and so such CS disaccharide forms may, if desired, be excluded from the summing of CS disaccharide forms that is done to determine or measure Total CS concentration.

In embodiments of the invention where the total concentration of CS is measured as one of the GAG properties, then it may be preferred that at least one other GAG property or CS property in accordance with the invention is measured, e.g. a property that is not based on the total level of the other individual GAGs present (e.g. not total HS). In some embodiments the total concentration of CS is not measured. In some embodiments, the measurement of one or more CS GAG properties in accordance with the invention is preferred.

“Charge CS” is another GAG form or property which may be measured in the present invention, e.g. as part of the GAG profile. “Charge CS” refers to the total fraction of sulfated disaccharides of CS, i.e. the fraction of sulfated disaccharides of CS present or measured or detected in a sample out of the total CS disaccharides present or measured or detected in a sample (i.e. sulfated CS disaccharides/sulfated+unsulfated CS disaccharides). In some embodiments, “Charge CS” refers to the weighted sum of the concentration of all CS disaccharides divided by the total CS, where the weight is the count of sulfo groups in that disaccharide, i.e. 0 for 0S CS, 1 for 4S CS, 6S CS, and 2S CS, 2 for 2S6S CS, 4S6S CS, and 2S4S CS, and 3 for Tris CS (and this is the definition of “Charge CS” used in connection with the term “Charge CS” in the Example section herein).

As the measurement of “charge CS” is dependent on the measurement of other properties, i.e. the measurement of levels of sulfated and unsulfated CS disaccharides, this property is not referred to herein as an independent GAG property or CS property.

In some embodiments it is preferred to measure up to 8 (e.g. 1, 2, 3, 4, 5, 6, 7 or 8) or all 8 of the CS sulfation forms (i.e. the sulfated and unsulfated forms), together with total CS. Charge CS may additionally be measured in some embodiments. In preferred embodiments, at least one (or at least 2, 3, 4, 5, 6, 7 or 8) CS sulfation form is measured.

In some embodiments, one or more (or all) of the following GAG properties may be measured or determined: the relative level of 4S CS with respect to 6S CS (e.g. the ratio 4S CS/6S CS or the inverse ratio 6S CS/4S CS), the relative level of 6S CS with respect to 0S CS (e.g. the ratio 6S CS/0S CS or the inverse ratio 0S CS/6S CS) or the relative level of 4S CS with respect to 0S CS (e.g. the ratio 4S CS/0S CS or the inverse ratio 0S CS/4Ss CS). In some embodiments, the relative level of 4S CS with respect to 6S CS (e.g. the ratio 4S CS/6S CS or the inverse ratio 6S CS/4S CS) is not measured or determined.

For HS, there are 8 main sulfated and unsulfated forms (sulfation patterns, disaccharide sulfation forms) which are: 0S HS (also referred to as unsulfated HS), 2S HS (which is sulfated at the 2-position of GlcA), NS HS (which is sulfated at the N-position of the GlcNAc), 6S HS (which is sulfated at the 6-position of the GlcNAc), 2S6S HS (which is sulfated at the 2-position of GlcA and the 6-position of the GlcNAc), NS6S HS (which is sulfated at the 6-position and N-position of GlcNAc), NS2S HS (which is sulfated at the 2-position of GlcA and the N-position of GlcNAc), Tris HS (which is sulfated at the 2-position of GlcA and 6-position and N-position of GlcNAc, also referred to as trisulfated HS). Note that sulfation in position 3 of the GlcNAc is also possible but rarely observed.

Each of the above is a form of HS GAG (an HS GAG form or property or feature or GAGome feature) which may be measured or determined in the methods of the present invention. However, due to its rarity, in preferred embodiments of the invention, the sulfation form with sulfation in position 3 of the GlcNAc is not measured. Thus, in the methods of the invention, one or more (or all) of these 9 (or preferably 8) forms may be measured, for example up to 9 (or preferably up to 8), e.g. 1, 2, 3, 4, 5, 6, 7, 8 or all 9 of these sulfation forms may be measured. In some embodiments, measurement of all 8 of these sulfation forms (excluding the sulfation form with sulfation in position 3 of the GlcNAc) is preferred.

Another GAG property for HS which may be measured in the methods of the present invention is the total concentration of HS (which may also referred to as HS tot or Tot HS or total HS) or the total level of HS. This is typically measured as a concentration, e.g. an absolute concentration, e.g. in μg/ml, as described elsewhere herein. In some embodiments, the total HS is measured by summing the level of all measured HS disaccharide forms listed above (preferably the 8 main sulfated and unsulfated forms, i.e. excluding the rare sulfation form with sulfation in position 3 of the GlcNAc). Thus, total HS may be the sum of the levels of all measured HS disaccharide forms listed above (preferably the sum of the 8 main sulfated and unsulfated forms, i.e. excluding the rare sulfation form with sulfation in position 3 of the GlcNAc). In some embodiments, the total HS is measured by summing the level of all of the HS disaccharide forms that are measured at (or above) an analytically detectable level, or at (or above) a level that has been selected as a minimum threshold level (a minimum threshold level that is considered analytically detectable or analytically meaningful, for example, the limit of detection or the lower limit of quantification for such HS disaccharide form). For example, the total HS may be the sum of all of the HS disaccharide forms that are measured at a level at, or above, a given analytically detectable level, or at, or above, a selected minimum threshold level (e.g. at a concentration of >0.1 μg/ml). The level of HS disaccharide forms below a given analytically detectable level or below a minimum threshold level would typically provide only a negligible (or de minimis) contribution to the overall HS concentration, and so such HS disaccharide forms may, if desired, be excluded from the summing of HS disaccharide forms that is done to determine or measure Total HS concentration.

In embodiments of the invention where the total concentration of HS is measured as one of the GAG properties, then it may be preferred that at least one other GAG property or HS property in accordance with the invention is measured, e.g. a property that is not based on the total level of the other individual GAGs present (e.g. not total CS). In some embodiments the total concentration of HS is not measured. In some embodiments, the measurement of one or more HS GAG properties in accordance with the invention is preferred.

“Charge HS” is another GAG form or property which may be measured in the present invention, e.g. as part of the GAG profile. “Charge HS” refers to the total fraction of sulfated disaccharides of HS, i.e. the fraction of sulfated disaccharides of HS present or measured in a sample out of the total HS disaccharides present or measured in a sample (i.e. sulfated HS disaccharides/sulfated+unsulfated HS disaccharides). In some embodiments, “Charge HS” refers to the weighted sum of the concentration of all HS disaccharides divided by the total HS, where the weight is the count of sulfo groups in that disaccharide, i.e. 0 for 0S HS, 1 for NS HS, 6S HS, 2S HS, 2 for 2S6S HS, NS6S HS, and NS2S HS, and 3 for Tris HS (and this is the definition of “Charge HS” used in connection with the term “Charge HS” in the Example section herein).

As the measurement of “charge HS” is dependent on the measurement of other properties, i.e. the measurement of sulfated and unsulfated HS disaccharides, this property is not referred to herein as an independent GAG property or HS property.

In some embodiments it is preferred to measure up to 8 (e.g. 1, 2, 3, 4, 5, 6, 7 or 8) or all 8 of the HS main sulfation forms (i.e. the sulfated and unsulfated forms listed above excluding the sulfation form with sulfation in position 3 of the GlcNAc), together with total HS. Charge HS may additionally be measured in some embodiments. In preferred embodiments, at least one (or at least 2, 3, 4, 5, 6, 7 or 8) HS sulfation form is measured.

In some embodiments, the 8 main sulfated and unsulfated HS forms, total HS, the 8 main sulfated and unsulfated CS forms, and total CS are measured, i.e. 18 GAG properties.

In some embodiments, the 8 main sulfated and unsulfated HS forms and the 8 main sulfated and unsulfated CS forms are measured, i.e. 16 GAG properties.

In some embodiments, the level and/or chemical composition of hyaluronic acid (HA) may be additionally determined in a body fluid sample. Hyaluronic acid (HA) is typically non-sulfated. Accordingly, when HA is measured in accordance with the invention, it is typically and preferably the level (total level or total concentration) of HA that is measured (which may also be referred to as Total HA). This is typically measured as a concentration, e.g. in μg/ml, as described elsewhere herein. In some embodiments, HA is not measured.

In some embodiments, the 8 main sulfated and unsulfated HS forms, total HS, the 8 main sulfated and unsulfated CS forms, total CS and total HA are measured, i.e. 19 GAG properties.

CS (total CS) and HS (total HS) is typically measured in terms of an absolute concentration, e.g. in μg/ml, as described elsewhere herein.

The various CS sulfation forms and HS sulfation forms may also be measured in terms of an absolute concentration, e.g. in μg/ml. Thus, in some embodiments the level (or concentration) of a given CS sulfation form or a given HS sulfation form is an absolute level or absolute concentration of a given CS sulfation form or a given HS sulfation form. In some embodiments, absolute levels or absolute concentrations are preferred.

However, the various CS sulfation forms and HS sulfation forms may alternatively, or additionally, be measured in terms of a relative concentration (or relative level). Thus, in some embodiments the level (or concentration) of a given CS sulfation forms or a given HS sulfation form is the relative level or relative concentration.

Thus the “level” or “concentration” of GAG sulfation forms may be its absolute concentration or its relative concentration. In some embodiments, both the absolute concentration and the relative concentration of one or more sulfated GAG forms is measured (or determined).

The “relative concentration” may be considered the mass fraction (e.g. in %) of a given CS sulfation form or a given HS sulfation form that is obtained (or calculated or determined) by normalizing its absolute concentration to the total concentration of the relevant GAG class, i.e. by normalizing its absolute concentration to the total CS concentration or total HS concentration (as appropriate).

Thus, the relative concentration of a given CS sulfation form may be considered the mass fraction (e.g. in %) of said given CS sulfation form that is obtained (or calculated or determined) by normalizing its absolute concentration by the total CS concentration.

The relative concentration of a given HS sulfation form may be considered the mass fraction (e.g. in %) of said given HS sulfation form that is obtained (or calculated or determined) by normalizing its absolute concentration by the total HS concentration.

Relative concentration may be expressed in terms of a percentage (%).

Thus, in some embodiments the level (or concentration) of a given CS sulfation form or a given HS sulfation form may be the absolute concentration and/or the relative concentration of the given CS sulfation form or the given HS sulfation form. In some embodiments the level (or concentration) of a given CS sulfation form or a given HS sulfation form is the absolute concentration of the given CS sulfation form or the given HS sulfation form. In some embodiments the level (or concentration) of a given CS sulfation form or a given HS sulfation form is the relative concentration of the given CS sulfation form or the given HS sulfation form.

A relative concentration may be alternatively viewed as a “fraction” or “mass fraction” or “proportion” or “relative measurement” as discussed below.

As discussed above, GAG properties or GAG forms, e.g. disaccharide sulfation forms (with the exception of total CS or total HS) may be measured as a fraction size or fraction or mass fraction (e.g. μg/μg) or proportion or relative measurement, rather than as absolute levels or concentrations, for example are given a value of less than 1 or are normalised to 1 depending on the levels of all the sulfation forms for the relevant GAG class (or all the main sulfation forms for the relevant GAG class) measured in the sample (or are expressed in terms of a %). In other words, the level of each of the desired sulfation forms is measured independently and then normalised to 1. In other words, the level of each of the desired sulfation forms is measured independently and then its mass fraction or volume fraction or mole fraction is computed. These fractions may also be expressed as percentage. In other words, these fractions may also be normalised to 100. For example, in some embodiments, the fraction size of a given sulfated CS form or unsulfated CS form may be determined by measuring the level of the given sulfated CS form or unsulfated CS form and dividing this by the sum of the levels of all of the CS sulfation forms (or all of the main sulfation forms) and the unsulfated CS form measured (or present) in the sample. In some embodiments, the fraction size of a given sulfated HS form or unsulfated HS form may be determined by measuring the level of the given sulfated HS form or unsulfated HS form and dividing this by the sum of the levels of all of the HS sulfation forms (or main sulfation forms) and the unsulfated HS form measured (or present) in the sample. When calculating such fractions, it is preferred that at least the main sulfation forms of CS or HS are measured in order to be able to normalise the fraction of the particular individual sulfation form to 1.

Relative measurements may be easier to interpret, for example, a measurement of 0s HS of 0.6 indicates that 60% of the measured HS disaccharides are unsulfated. However, absolute levels can also be measured. Indeed, in some embodiments it is preferred to measure absolute concentrations of one or more sulfation forms.

In some preferred embodiments of the invention, the disaccharide composition (for example the specific sulfation patterns (e.g. sulfation forms)) of one or more (or all) of the disaccharides making up CS and/or HS is measured or determined. In some embodiments one or more (or all) sulfation properties or forms (or GAG features or properties or GAGome features or properties) of CS and/or HS such as those outlined above (e.g. 0S CS, 2S CS, etc), are measured or determined. Appropriate methods of doing this would be well known to a skilled person in the art and any of these could be used. However, a convenient method to achieve such quantification of disaccharide composition or the appropriate properties or forms of CS or HS (and separation of the disaccharide forms) is to use electrophoresis, in particular capillary electrophoresis, e.g. capillary electrophoresis with fluorescence detection, e.g. capillary electrophoresis with laser-induced fluorescence detection (CE-LIF). An alternative method is liquid chromatography, preferably HPLC (high-performance liquid chromatography), for example SAX HPLC. Preferably mass spectrometry is also used (e.g. HPLC-MS), for example electrospray ionization mass spectrometry (ESI-MS). Alternatively, mass spectrometry can be used without chromatography, e.g. liquid chromatography. One example is capillary electrophoresis with laser-induced fluorescence detection. Another example is HPLC ESI-MS. Preferred methods involve high performance liquid chromatography (HPLC), preferably ultra-HPLC (UHPLC), in combination with mass spectrometry, e.g. MS/MS or triple quadrupole mass spectrometry. Preferred methods comprise ultra-high-performance liquid chromatography (UHPLC) coupled with electrospray ionization triple-quadrupole mass spectrometry. Particularly preferred methods are outlined in the Examples.

In some methods of the invention where the levels of one or more individual disaccharide forms are measured, the GAGs are subjected to a processing step, for example a step of fragmentation or cleavage or digestion, e.g. by chemical digestion or enzyme treatment in order to obtain the disaccharide units which are then analysed. The enzyme may be a GAG lyase, e.g. a chondroitinase or a heparinase, or a combination of chondrotinases, or a combination of heparinases, or a combination of one or more chondroitinases and one or more heparinases. Preferably, the chondroitinase is Chondroitinase ABC or Chondroitinase AC or Chondroitinase A or Chondroitinase B or Chondroitinase C. Preferably the heparinase is Heparinase I-II-III. In preferred embodiments one or more chondroitinases and one or more heparinases are used, preferably Chondroitinase ABC and Heparinase I-II-III.

In some methods of the invention the GAGs in the sample are subjected to a step of extraction (e.g. using a proteolytic agent such as a protease, e.g. a non-specific protease, e.g. proteinase K) and/or purification, e.g. using an anion-exchange resin (or other means to purify GAGs based on the negative charge of the GAGs).

However, in preferred methods of the invention one or both of these steps is not carried out (i.e. there is no such extraction and/or no such purification). For example, in preferred embodiments of the invention, e.g. where the level and/or composition of the protein-free fraction of one or more GAGs is determined, then no such protein digestion (extraction) step is carried out. In other words, said methods do not involve a processing step in which samples are contacted with a proteolytic agent, such as for example a protease, e.g. proteinase K. As discussed elsewhere herein, omitting a processing step in which samples are contacted with a proteolytic agent means that the protein-free fraction of GAGs can be specifically analysed.

In other preferred embodiments of the invention, said methods do not involve a processing step in which the GAGs (e.g. one or both of the GAGs CS and HS) are purified from the sample based on the negative charge of said GAGs, e.g. using an anion-exchange resin. Without wishing to be bound by theory, omitting a processing step in which the GAGs (e.g. one or both of the GAGs CS and HS) are purified from the sample based on the negative charge of said GAGs (e.g. using an anion-exchange resin) simplifies the method and can lead to efficiencies in terms of the yield of GAGs obtained during processing of the body fluid sample.

In preferred methods of the present invention, methods do not involve a processing step in which samples are contacted with a proteolytic agent, such as for example a protease, e.g. proteinase K, and do not involve a processing step in which the GAGs (e.g. one or both of the GAGs CS and HS) are purified from the sample based on the negative charge of said GAGs, e.g. using an anion-exchange resin (or other means to purify GAGs based on the negative charge of the GAGs).

In some methods of the invention the GAGs in the sample (e.g. various different GAG forms in the sample) are subjected to a step of separation and/or quantification, as described elsewhere herein. For example, as discussed elsewhere herein, HPLC in combination with mass spectrometry may be used in preferred embodiments. Particularly preferred methods comprise ultra-high-performance liquid chromatography (UHPLC) coupled with electrospray ionization triple-quadrupole mass spectrometry.

Other methods which might be used are known in the art. However, examples are analytical techniques involving the use of antibodies to various GAG forms, e.g. techniques such as Western blot, ELISA or FACS, or methods involving agarose gel electrophoresis (e.g. fluorophore-assisted carbohydrate electrophoresis (FACE)) or polyacrylamide gel electrophoresis (PAGE).

In any embodiments which refer to the determination of the level of one or more from a certain list of GAG forms (or GAG properties or GAG features or GAGome features) in accordance with the invention, in some such embodiments the level of all the listed GAG forms (or GAG properties or GAG features or GAGome features) in accordance with the invention may be determined.

In some embodiments, the level of a single GAG form (GAG property or GAG feature or GAGome feature) is determined.

In other embodiments of the present invention, the level of more than one GAG form (GAG property) is determined (e.g. the level of two or more GAG forms, or three or more GAG forms, or four or more GAG forms, or five or more GAG forms is determined). By “more than one” is meant 2, 3, 4, 5, 6, 7, 8, 9, 10, 11, 12, 13, etc. In any list of markers or GAG properties provided herein, in some embodiments all are measured (or determined). Also, a determination of the level of each and every possible combination of the GAG forms can be performed.

Thus, in some embodiments multi-marker methods are performed. Determining the level of multiple of the GAG forms (biomarker multiplexing) in accordance with the invention may improve screening (e.g. diagnostic) accuracy.

Thus, although markers (GAG forms or GAG properties or features or GAGome features) in accordance with the invention can be used in the methods of the invention individually, they can also be used in combination, e.g. in the form of a multi-marker assay.

In some embodiments, the level of a single GAG form (GAG property) in accordance with the invention is used for the basis of the screening for OA or RA, e.g. discrimination (or distinguishing) between OA and RA may be made on the basis of the level of a single GAG form in accordance with the invention in some embodiments (i.e. on the basis of the level of 4S CS, NS HS, 0S HS, total concentration of CS or total concentration of HS). In other embodiments, more than one (e.g. 2, 3, 4 or 5) GAG form in accordance with the invention is used for the basis of the screening for OA or RA, e.g. a discrimination (or distinguishing) between OA and RA may be made on the basis of the level of more than one GAG form in accordance with the invention (i.e. on the basis of the level of more than one (e.g. all) of 4S CS, NS HS, 0S HS, total concentration of CS or total concentration of HS, e.g. any sub-groups of these GAG forms as discussed elsewhere herein.

In some embodiments, where one GAG form or a group (or sub-group or subset) of GAG forms in accordance with the invention is used for the basis of the screening for OA or RA (e.g. to discriminate or distinguish between OA and RA), the level of one or more (or all) of the other GAG forms (or GAG properties) described herein may be additionally determined or measured.

Based on the observed alterations in the levels of various GAG properties in accordance with the invention, if desired, scoring methods, scoring systems, markers or formulas can be designed which use such levels of various GAG forms in order to arrive at an indication, e.g. in the form of a value or score, which can then be used for screening (e.g. diagnosis, etc.). Appropriate scoring systems and parameters (e.g. GAG forms) to be measured can readily be designed based on (or including) one or more of the individual GAG features in accordance with the present invention.

In some embodiments in accordance with the present invention, the chemical composition may be expressed in terms of score (or GAG score), said score being based on (or derived or calculated by using) the measured level of one or more (preferably more than one) of the GAG properties in accordance with the invention.

Thus, a score may be based on (or derived or calculated or computed using) one or more (or all) measured (or determined) GAG properties selected from the group consisting of: 4S CS, NS HS, 0S HS, total CS and total HS.

In some embodiments, a score may be based on (or derived or calculated or computed using) measured levels of one or more GAG properties in more than one type of body fluid sample. In some such embodiments, the GAG properties measured in each type of body fluid may be the same or they may be different.

In some embodiments, appropriate threshold or cut-off values (e.g. used to declare a sample as being OA or RA (or indicate likely OA or likely RA), as appropriate, for use with scores can be designed by a person skilled in the art. By way of example, a cut-off (or threshold) value may be calculated based on ROC curves. In some cases, maximally selected rank statistics may be used to identify a cut-off value (or cut-off score).

In some embodiments, screening (e.g. diagnosis, etc.) may be done by comparing a given score for a sample (or subject) to be screened (e.g. diagnosed) with a threshold or cut-off value, and assigning a result (e.g. an indication of OA or RA) based on whether the score determined is above or below (e.g. significantly above or significantly below) a cut-off value.

Any scoring methods, scoring systems, markers or formulas can be used that comprise any appropriate combination of the GAG properties in accordance with the invention in order to arrive at an indication, e.g. in the form of a value or score, which can then be used for screening (e.g. diagnosis) of OA or RA. For example, said methods etc., can be an algorithm that comprises any appropriate combination of the GAG properties in accordance with the invention as input, to e.g. perform pattern recognition of the samples, in order to arrive at an indication, e.g. in the form of a value or score, which can then be used for screening (e.g. diagnosis) of OA or RA. Non-limiting examples of such algorithms include machine learning or deep learning algorithms that implement classification (algorithmic classifiers), such as linear classifiers (e.g. Fisher's linear discriminant, logistic regression, naive Bayes classifier, perceptron); support vector machines (e.g. least squares support vector machines); quadratic classifiers; kernel estimation (e.g. k-nearest neighbor); boosting; decision trees (e.g. random forests); neural networks; learning vector quantization.

The use of such classifiers, e.g. machine learning classifiers, e.g. random forest classifiers, would be within the skill of a person skilled in the art.

In some embodiments, a multivariable logistic regression model may be used to compute the GAG score.

As described elsewhere herein, screening for OA or RA in accordance with the present invention may involve using a score, or expressing the level and/or chemical composition in accordance with the invention using a score. As described elsewhere herein, in some such embodiments, an altered score (e.g. increased or decreased as the case may be) in comparison to a control score (or cut-off level or threshold level) is indicative of OA or RA (as appropriate) in said subject.

As discussed above, the present invention provides a method of screening for OA or RA in a subject. The present invention also provides a method of diagnosing OA or RA in a subject. Thus, the method of screening for OA or RA in accordance with the present invention can be used, for example, for diagnosing OA or RA.

Thus, in one aspect the present invention provides a method for diagnosing OA or RA in a subject. In some embodiments, a positive diagnosis (i.e. the presence of OA or RA as the case may be) is made if the level of one or more of the GAG properties in accordance with the invention in the sample is altered (increased or decreased as the case may be) in comparison to an appropriate control level (e.g. as discussed elsewhere herein).

Thus, a further aspect, the present invention provides a method of diagnosing osteoarthritis (OA) or rheumatoid arthritis (RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

Methods of screening (e.g. diagnosis) in accordance with the present invention may be used to discriminate between (or distinguish between or differentiate between) OA or RA in a subject having arthritis or suspected of having arthritis.

Thus, in another aspect, the present invention provides a method of discriminating between (or distinguishing between or differentiating between) OA and RA in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

In a preferred aspect, the present invention provides a method of discriminating between (or distinguishing between or differentiating between) osteoarthritis (OA) and rheumatoid arthritis (RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a urine sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject. Embodiments of methods of screening of the invention described elsewhere herein may be applied, mutatis mutandis, to this aspect of the invention (e.g. preferred GAG forms (or groups of GAG forms), preferred processing steps, preferred control levels, etc.).

The level of one or more GAG property in accordance with the present invention may be used in (or for) the differential diagnosis of OA and RA (OA versus RA), or as a part of a differential diagnosis process for OA and RA (OA versus RA).

The concept of “differential diagnosis” is well understood in the art. Briefly, differential diagnosis can be considered a process in which a differentiation is made between two or more diseases (or conditions) that could be causing a subject's symptoms. Thus, a differential diagnosis process can be used when a subject's symptoms match symptoms that can be caused by more than one condition.

Thus, in another aspect, the present invention provides a method of differential diagnosis OA or RA (OA versus RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

As described elsewhere herein, methods of screening (e.g. diagnosis) in accordance with the present invention may be used in conjunction with other diagnostic tests or methods.

Thus, in another aspect, the present invention provides a method of confirming (or validating) a prior diagnosis of OA or RA in a subject, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

In another aspect, the present invention provides a method of screening for osteoarthritis (OA) or rheumatoid arthritis (RA) in a subject having arthritis or in a subject suspected of having arthritis, said method comprising:
determining the level of one or more GAG property selected from the group consisting of: (i) 4S CS, (ii) NS HS, (iii) 0S HS, (iv) total concentration of CS, and (v) total concentration of HS in a body fluid (e.g. urine) sample,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

In another aspect, the present invention provides a method of discriminating between osteoarthritis (OA) and rheumatoid arthritis (RA) in a subject having arthritis or in a subject suspected of having arthritis, said method comprising:
determining the level of one or more GAG property selected from the group consisting of: (i) 4S CS, (ii) NS HS, (iii) 0S HS, (iv) total concentration of CS, and (v) total concentration of HS in a body fluid (e.g. urine) sample,
wherein said sample has been obtained from said subject. Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

In another aspect, the present invention provides a method of diagnosing osteoarthritis (OA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

In a further aspect, the present invention provides a method of providing information that is useful in (or for) screening (e.g. diagnosing, differentially diagnosing, discriminating between or confirming a diagnosis of) osteoarthritis (OA) or rheumatoid arthritis (RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject. Said information is of course typically the determined (or measured) level of one or more of said GAG properties.

Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

A yet further aspect of the invention provides a method of screening for (e.g. diagnosing, differentially diagnosing, discriminating between, or confirming a diagnosis of) OA or RA in a subject, said method comprising analysing disaccharide units that have been derived from one or both of the glycosaminoglycans (GAGs) chondroitin sulfate (CS) and heparan sulfate (HS) in a body fluid sample, wherein said method comprises the level of one or more GAG property in accordance with the invention. Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

A yet further aspect of the invention provides a method of screening (e.g. diagnosing, differentially diagnosing, discriminating between, or confirming a diagnosis of) OA or RA in a subject, said method comprising analysing a population of disaccharide units consisting essentially of disaccharide units that have been derived from the non-proteoglycan fraction (or protein-free fraction) of one or both of the glycosaminoglycans (GAGs) chondroitin sulfate (CS) and heparan sulfate (HS) in a body fluid sample, wherein said method comprises the level of one or more GAG property in accordance with the invention. Embodiments of methods of screening of the invention described elsewhere herein may be applied, mutatis mutandis, to this aspect of the invention (e.g. preferred GAG forms (or groups of GAG forms), preferred processing steps, preferred body fluid, preferred control levels, etc.).

Unless otherwise clear from the context, the features and discussion herein in relation to methods of screening for OA or RA (e.g. in relation to preferred GAG properties or combinations thereof, or scores for measurement, preferred processing steps, preferred body fluid, preferred control levels, etc.) apply, mutatis mutandis, to the other related methods of present invention (to a method of diagnosing, differentially diagnosing, discriminating between, or confirming a diagnosis of, method of providing information useful for OA or RA, etc.).

In some embodiments, the invention provides the use of the methods of the invention (e.g. screening, diagnostic or methods of determining progression, etc., as described herein) in conjunction with other known screening, diagnostic or progression monitoring methods for OA or RA (as appropriate), such as radiological imaging (e.g. computed tomography, CT, or positron emission tomography, PET, scan, or X-ray) or magnetic resonance imaging (MRI scan) or ultrasound imaging, or histological assessment (e.g. using a biopsy), or a set of criteria that has been established to be indicative of (e.g. diagnostic of) OA or RA. By way of an example, in the case of OA or RA, radiological imaging may be used in conjunction with a method of the present invention. By way of another example, for OA or RA, other screening or diagnostic criteria for OA or RA (e.g. in the case of RA the American Rheumatism Association criteria for RA) may be used in conjunction with a method of the present invention. In the case of OA, the KL (Kellgren-Lawrence) system (or KL scoring system) may be used in conjunction with a method of the present invention. The KL system is a well-known radiographic classification system for OA.

Thus, for example, methods of the invention can be used to confirm a diagnosis (confirm a prior diagnosis) of OA or RA in a subject. In some embodiments the methods of the present invention are used alone.

The level of the GAG form in question can be determined or measured by analyzing the sample which has been obtained from or removed from the subject by an appropriate means. The determination is typically carried out in vitro.

Levels of one or more of the GAG forms in the sample can be measured (determined) by any appropriate assay or technique or method, a number of which are well known and documented in the art. Electrophoresis, e.g. agarose gel electrophoresis or capillary electrophoresis (in particular capillary electrophoresis with fluorescence detection such as CE-LIF) is a technique that can be used for measuring (determining) the levels of one or more of the GAG forms in accordance with the invention. Liquid chromatography, in particular HPLC (high-performance liquid chromatography) in combination with mass spectrometry (MS) are preferred techniques for measuring (determining) the levels of one or more of the GAG forms in accordance with the present invention.

Suitable electrophoresis, e.g. capillary electrophoresis, and liquid chromatography, e.g. HPLC techniques for GAG form analysis, together with appropriate mass spectrometry methods (and associated data processing techniques) are well known and documented in the art.

One method that may be used in the invention is capillary electrophoresis with laser-induced fluorescence detection, CE-LIF (e.g. as described in Galeotti et al., 2014, Electrophoresis 35:811-818; and Kottler et al., 2013, Electrophoresis 34:2323-2336). HPLC combined with post column derivatization and fluorimetric detection can also be used, e.g. as described in Volpi 2006, Curr Pharm Des 12:639-658, as can HPLC combined with ESI-MS (electrospray ionization-mass spectrometry), e.g. as described in Volpi and Linhardt, 2010, Nature protocols 5:993-1004, also with fluorimetric detection, e.g. as described in Galeotti and Volpi, 2011, Anal Chem 83:6770-6777, or Volpi et al., 2014, Nature Protocols 9:541-558. Agarose gel electrophoresis can also be used, e.g. FACE (fluorophore assisted carbohydrate electrophoresis) as described in Volpi and Maccari, 2006, Analyt Technol Biomed Life Sci, 834:1-13; and Volpi and Maccari, 2002, Electrophoresis 23:4060-4066.

A particularly preferred method for determining the level of one or more of the GAG forms in the sample is described herein in the Examples. Thus, preferred methods may involve high performance liquid chromatography (HPLC), preferably ultra-HPLC (UHPLC), in combination with mass spectrometry, e.g. MS/MS or triple quadropole mass spectrometry. Particularly preferred methods comprise ultra-high-performance liquid chromatography (UHPLC) coupled with electrospray ionization triple-quadrupole mass spectrometry system. An example of such methods is described in Tamburro et al. (Journal of Chromatography B, 1177 (2021) 122761).

Certain methods of sample preparation (or processing), e.g. GAG extraction and purification are also known and described in the art, for example Volpi and Maccari, 2005, Biomacromolecules 6:3174-3180 and Clin Chim Acta 356:125-133, Coppa et al., 2011 Glycobiology 21:295-303. Such reported art based methods of sample preparation (or processing) involve a protease treatment (protease extraction) and purification step based on using an anion-exchange resin. In some methods of the present invention such a protease treatment step and/or purification step using an anion-exchange resin may be performed. However, as discussed elsewhere herein, in preferred methods a step of protease treatment and/or a purification step using an anion-exchange resin is not performed. In particular, in preferred methods where the protein-free fraction of the GAGs is analysed, then a step of protease treatment is not performed.

In some embodiments HPLC and mass spectrometry (and associated data processing techniques) is used to obtain a fraction of the level of one or more particular GAG forms (e.g. the sulfated or unsulfated disaccharide forms) in the sample in comparison to the total amount. For example, after sample preparation, GAGs can be digested using enzymes, separated in an HPLC column and characterized using MS. As described elsewhere herein, the quantities of one or more individual GAG forms (e.g. a particular sulfated or unsulfated disaccharide form) may be conveniently normalised (i.e. divided) by the sum of all the quantities of individual GAG forms measured, to yield fractions (or proportions or relative concentrations). However, absolute concentrations (or absolute levels) of individual GAG forms (e.g. GAG sulfation forms) may alternatively, or additionally, be measured.

In accordance with the present invention, a quantitative, semi-quantitative or qualitative assessment (determination) of the level of one or more of the GAG forms can be made.

Appropriate methods of doing this would be well known to a skilled person in the art and any of these could be used. However, a convenient method to achieve such quantification of disaccharide composition or the appropriate properties or forms of CS or HS (and separation of the disaccharide forms) is to use electrophoresis, in particular capillary electrophoresis, e.g. capillary electrophoresis with fluorescence detection, e.g. capillary electrophoresis with laser-induced fluorescence detection (CE-LIF) (e.g. as described in Galeotti 2014, supra, or Kottler 2013, supra). An alternative method, which is preferred in some embodiments, is to use liquid chromatography, preferably HPLC (high-performance liquid chromatography), for example SAX HPLC or for example as described in Volpi 2006, supra, Galeotti and Volpi 2011, supra, Volpi et al., 2014, supra or Volpi and Linhardt, 2010, supra. Preferably mass spectrometry is also used (HPLC-MS), for example electrospray ionization mass spectrometry (ESI-MS), e.g. HPLC ESI-MS. Particularly preferred methods are outlined in the Examples. One example of a particular method is capillary electrophoresis (e.g. for example, capillary electrophoresis with laser-induced fluorescence detection). Another particularly preferred example would be HPLC followed by MS (HPLC-MS), e.g. HPLC ESI-MS. Preferred HPLC-MS methods are discussed elsewhere herein.

Thus, in preferred methods of the invention said level or chemical composition of said GAG or GAG property is determined by HPLC and mass spectrometry. Preferably said HPLC is ultra-HPLC and/or said mass spectrometry is triple-quadrupole mass spectrometry. In certain preferred methods, said level or chemical composition of said GAG or GAG property is determined by high performance liquid chromatography (HPLC), preferably ultra-HPLC (UHPLC), in combination with mass spectrometry, e.g. MS/MS or triple quadrupole mass spectrometry. Preferred methods comprise ultra-high-performance liquid chromatography (UHPLC) coupled with (or in combination with) electrospray ionization triple-quadrupole mass spectrometry.

Generally, the determination of the GAG properties (or forms or features) in accordance with the present invention does not involve the measurement of GAG molecules in the exact same form as found in the body fluid of a subject (e.g. does not involve the measurement of a naturally occurring form of GAG). For example, such native or naturally occurring GAG molecules are often found in biological samples, e.g. body fluid samples, in the form of long sugar chains which can either be attached to proteins (also referred to herein as protein-bound GAGs or proteoglycan GAGs), or not attached to proteins (also referred to herein as free GAGs or protein-free GAGs).

In some embodiments, methods of the invention may include a step of processing a sample. In some embodiments, the methods of the invention may thus be performed on such processed samples or materials derived from such processed samples. Thus, generally the methods of the invention are carried out on samples which have been processed in some way (e.g. are man-made rather than native samples).

Processing steps may include, but are not limited to, extraction or purification of GAGs from the sample, steps of fragmentation or cleavage or digestion of proteins present in the sample, e.g. as a means of separating or extracting or removing GAGs from the protein to which they are attached, e.g. through the use of a protease such as proteinase K, purification of GAGs, e.g. using an anion-exchange resin, isolating cells from the sample, isolating cell components from the sample, extracting (e.g. isolating or purifying) proteins/peptides from the sample. Said processing steps thus also include steps carried out on a body fluid sample to prepare it for analysis, e.g. in the case of a urine sample, the removal of cells or other impurities may be done. A processing step may involve one or more of digestion, extraction, purification, boiling, filtration, lyophilization, fractionation, centrifugation, concentration, dilution, inactivation of interfering components, addition of reagents, derivatization, complexation and the like. Exemplary processing steps are described in the Examples.

Although in certain methods of the invention steps of fragmentation or cleavage or digestion of proteins present in the sample (e.g. as a means of separating or extracting or removing GAGs from the protein to which they are attached, e.g. through the use of a protease such as proteinase K) and/or purification of GAGs (e.g. using an anion-exchange resin) may be done, as is evident from the discussion elsewhere herein in certain preferred methods a step of fragmentation/cleavage/digestion of proteins and/or a step of purification (e.g. using an anion-exchange resin) is not performed. In particular, in preferred methods where the level and/or chemical composition of the protein-free fraction of the GAGs is determined, a step of fragmentation/cleavage/digestion of proteins is not performed.

In general, the GAG containing body fluid sample that has been obtained from a subject is subjected to at least one processing step prior to determining the level and/or chemical composition in accordance with the methods of the present invention. In particular, in methods wherein the levels of one or more of the specific sulfated or unsulfated forms of CS or HS disaccharides are determined, the GAGs are preferably subjected to a processing step to obtain the disaccharide units for analysis.

In some such methods of the invention where the levels of certain individual disaccharide forms are measured, the GAGs, e.g. the full length GAG molecules, or polymerised polysaccharide chains of GAGs, or chains of repeating disaccharide units of GAGs, are subjected to a processing step, for example a step of fragmentation or cleavage or digestion, e.g. by chemical digestion or enzyme treatment. Appropriate methods of digestion or enzyme treatment would be known to a person skilled in the art, e.g. the use of one or more GAG lyase enzymes, e.g. one or more chondroitinase enzymes such as Chondroitinase ABC or Chondroitinase B, and/or the use of one or more heparinase enzymes such as Heparinase I-II-III, in order to obtain the disaccharide units which are then analysed.

Other methods to determine levels or compositions of GAGs which might be used are known in the art. However, examples are analytical techniques involving the use of antibodies to various GAG forms, e.g. techniques such as Western blot, ELISA or FACS, or methods involving agarose gel electrophoresis (e.g. fluorophore-assisted carbohydrate electrophoresis (FACE)) or polyacrylamide gel electrophoresis (PAGE).

In some embodiments, the level of one or more GAG forms (e.g. specific sulfated or unsulfated forms of CS or HS disaccharides, which have for example been derived from the full length GAG molecule or a chain of repeating disaccharide units of a GAG molecule by fragmentation, cleavage or digestion) in association with (e.g. physical association with or in complex with or derivatized with or labelled with) a reagent (e.g. 2-aminoacridone) that is being used to detect the GAG form is determined. Thus, in some embodiments the level of a complex of a GAG form and the reagent used to detect the GAG form is determined. Reagents suitable for detecting particular GAG forms are discussed elsewhere herein, but include antibodies, or some kind of fluorophore (or other detectable label or dye) attached to (or used to derivatize) the GAG form in question, for example to make it detectable by a fluorimeter (or other detection device). Thus, purely by way of example, in some embodiments the level of a GAG form in association with (e.g. in complex with or derivatized with) an antibody or fluorophore or the like may be determined. In some embodiments the level of a GAG form in association with (e.g. in complex with or derivatized with) 2-aminoacridone may be determined.

As certain preferred methods of the invention comprise the step of determining the level and/or chemical composition of the protein-free fraction of one or both of the glycosaminoglycans (GAGs) chondroitin sulfate (CS) and heparan sulfate (HS) in a body fluid sample, advantageously there is no need for the GAG molecules to be separated or extracted from the proteins to which they are attached. Instead, in such embodiments, the protein-free fraction (only the protein-free fraction) of the GAGs in a body fluid sample can be analysed from the sample without any need for such processing to separate the GAGs from the protein, e.g. by digesting the protein. Thus, preferred methods do not comprise a processing step in which said samples are contacted with a proteolytic agent such as a protease.

Other preferred methods do not comprise a step in which the GAGs are purified from the sample based on the negative charge of said GAGs (e.g. using an anion-exchange resin).

Thus, in preferred methods of the present invention, said sample has been obtained from said subject and has been subjected to processing prior to determining said level and/or chemical composition, wherein said processing
(a) comprises fragmenting said one or both GAGs into disaccharide units; and
(b) does not comprise prior to (a) at least one of:
   (i) contacting said sample with a proteolytic agent; and
   (ii) purifying said one or both GAGs in said sample based on the negative charge of said GAGs.

A yet further aspect of the invention provides a method of screening for OA or RA in a subject having arthritis, or in a subject suspected of having arthritis, said method comprising determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of (i) 4S CS, (ii) NS HS, (iii) 0S HS, (iv) total concentration of CS, and (v) total concentration of HS,
wherein said sample has been obtained from said subject and has been subjected to processing prior to determining said level and/or chemical composition,
wherein said processing
   (a) comprises fragmenting said one or both GAGs into disaccharide units; and
   (b) does not comprise prior to (a) at least one of:
      (i) contacting said sample with a proteolytic agent; and
      (ii) purifying said one or both GAGs in said sample based on the negative charge of said GAGs.

Embodiments of other aspects of the invention described elsewhere herein may be applied, mutatis mutandis, to this aspect of the invention (e.g. preferred GAG properties (or groups of GAG properties), preferred processing steps, preferred body fluid, etc.).

In preferred methods, said fragmenting of (a) is conveniently performed by contacting said one or both GAGs with one or more GAG lyase enzymes (e.g. as discussed elsewhere herein). For example, said fragmenting of (a) may be performed by contacting said one or both GAGs with one or more chondroitinase enzymes and/or one or more heparinase enzymes.

In art based methods, said contacting step of (b)(i) is conveniently performed by contacting said sample with one or more protease enzymes, e.g. proteinase K. Thus, in certain preferred methods of the invention such a step is not carried out. The proteolytic agent of (b)(i) may be a protease (e.g. a non-specific protease such as proteinase K). Thus, certain preferred methods of the invention do not comprise prior to (a) a step of contacting the sample with a protease (e.g. a non-specific protease e.g. proteinase K).

In art based methods, said purifying step of (b)(ii) is conveniently performed by using an anion-exchange resin. Thus, in certain preferred methods of the invention such a step is not carried out. Thus, certain preferred methods of the invention do not comprise prior to (a) a step of purifying said one or both GAGs in said sample using an anion-exchange resin.

In preferred methods of the invention, the method does not comprise the contacting of (b)(i).

In preferred methods of the invention, the method does not comprise the purifying of (b)(ii).

In other preferred methods of the invention neither step (b)(i) nor (b)(ii) are carried out. Thus, in particularly preferred embodiments the method does not comprise the contacting of (b)(i) or the purifying of (b)(ii).

As mentioned above, in some embodiments an altered level (increased or decreased as the case may be) of one or more of the GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) in comparison to a control level is indicative of either OA or RA in said subject (indicative of whether said subject has either OA or RA). The skilled person would be readily able to establish appropriate control levels for use in accordance with the invention.

In some embodiments of methods of the present invention (e.g, methods of screening or diagnosis or discriminating between),
(a) the level of one or more GAG property in accordance with the invention being different than (or altered (e.g. increased or decreased) in comparison to) a control level of the same one or more GAG property is indicative of OA, wherein said control level is within (or comprised within) a reference interval that has been determined for the same sample type obtained from a group of subjects (or from a reference population) not having OA; or
(b) the level of one or more GAG property in accordance with the invention being different than (or altered (e.g. increased or decreased) in comparison to) a control level of the same one or more GAG property is indicative of RA, wherein said control level is comprised within (or comprised within) a reference interval that has been determined for the same sample type obtained from a group of subjects (or from a reference population) not having RA.

In some embodiments, the group of subjects (or reference population) not having OA may be a group of subjects (or a reference population) having RA. In some embodiments, the group of subjects (or a reference population) not having RA may be a group of subjects (or a reference population) having OA.

The concept of “reference intervals” (which may also be referred to as “reference ranges”) is well known in the art, particularly in medicine and health related areas. A reference interval is the range (or interval) of values (e.g. levels) for a physiological measurement that has been established or determined for (or established or determined as being characteristic, or indicative of) a particular health state in a reference population (or reference group of subjects).

In some cases, a reference interval is the range (or interval) of values (e.g. levels) for a physiological measurement that has been established or determined for (or established or determined as being characteristic of, or indicative of) a particular disease (or condition) state in a reference population having a given disease or condition.

Reference intervals for a particular measurement or value (e.g. the level of one or more of the GAG properties in accordance with the invention) define an interval between which (or a range within which) a certain proportion (e.g. %) of measurements (or values) in a reference population fall into. The proportion may be for example 80% to 99%. For example the proportion may be 80%, 85%, 90% or 95%. Preferably, the proportion is 95%. Typically and preferably, the proportion is the central proportion of measurements (or values) in a reference population. By way of an example, when the proportion is 95% (preferably the central proportion is 95%), a reference interval for a particular measurement or value (e.g. the level of one or more of the GAG properties in accordance with the invention) is the interval between which (or range within which) 95% of the measurements (or values) for a reference population fall into. This means that 5% of measurements in such a reference population would be outside this reference interval. When the 95% proportion is the central proportion of the reference population (which is preferred), this means that 2.5% of measurements (or values) would be less than the lower limit of the reference interval and 2.5% of measurements (or values) would be higher than the upper limit of the reference interval. When an 80% proportion is the central proportion of the reference population, this means that 10% of measurements (or values) would be less than the lower limit of the reference interval and 10% of measurements (or values) would be higher than the upper limit of the reference interval. When an 85% proportion is the central proportion of the reference population, this means that 7.5% of measurements (or values) would be less than the lower limit of the reference interval and 7.5% of measurements (or values) would be higher than the upper limit of the reference interval. When a 90% proportion is the central proportion of the reference population, this means that 5% of measurements (or values) would be less than the lower limit of the reference interval and 5% of measurements (or values) would be higher than the upper limit of the reference interval.

The upper limit of a reference interval is typically referred to as an upper reference limit (URL). The lower limit of a reference interval is typically referred to as a lower reference limit or (LRL). In some embodiments, a URL or a LRL may be used a cut-off or threshold level to provide an indication of OA or RA. In some embodiments, an altered level (increased level or decreased level as the case may be) of one or more GAG property in accordance with the invention in comparison to (or relative to) a URL or a LRL of a reference interval is indicative of OA or RA (as appropriate).

The skilled person is familiar with reference intervals, and now that the present inventors have found that the levels of certain GAG properties are useful in screening for OA and RA in accordance with the present invention, the skilled person would readily be able to establish appropriate reference intervals (and associated URLs and LRLs) from (or of) appropriate reference populations for use in accordance with the present invention. As mentioned above, the concept of reference intervals is well-established in the art. For example, the Clinical and Laboratory Standards Institute publication “EP28-A3c Defining, Establishing, and Verifying Reference Intervals in the Clinical Laboratory; Approved Guideline—Third Edition” (2010, Vol. 28. No. 30; ISBN 1-56238-682-4) provides detailed discussion and guidance in this regard.

A reference interval may be based on measurements (or values) of (or taken from) any appropriate number of subjects in a reference population (i.e. the reference population may be of any appropriate size). The skilled person in readily able to select an appropriately sized reference population. Guidance on appropriate reference population sizes is provided in the above mentioned Clinical and Laboratory Standards Institute publication. Preferably, the reference population has at least 10 subjects. In some embodiments, there may be at least 10, at least 20, at least 30, at least 40, at least 50, at least 60, at least 70, at least 80, at least 90, at least 100, at least 120, at least 200, at least 500 subjects or at least 1000 subjects in a reference population. In some embodiments, there may be up to 50, up to 100, up to 200 or up to 500 subjects in a reference population. In some embodiments, there may be up to 10 to 20, 10 to 50, 10 to 100, 10 to 200, 10 to 500 or 10 to 1000 subjects in a reference population.

As is evident from the above, in some embodiments of the present invention that provide an indication of OA, a reference interval is a reference interval for a given GAG property in accordance with the invention that has been determined for the same sample type obtained from a reference population group of subjects not having OA (e.g. having RA). As is also evident from the above, in some embodiments of the present invention that provide an indication of RA, a reference interval is a reference interval for a given GAG property in accordance with the invention that has been determined for the same sample type obtained from a reference population group of subjects not having RA (e.g. having OA).

In some embodiments of methods of the present invention (e.g, methods of screening or diagnosis or discriminating between),
(a) the level of one or more GAG property in accordance with the invention being higher than (or increased in comparison to) a control level of the same one or more GAG property is indicative of OA, wherein said control level is within (or comprised within) a reference interval that has been determined for the same sample type obtained from a group of subjects (or from a reference population) not having OA; or
(b) the level of one or more GAG property in accordance with the invention being lower than (or decreased in comparison to) a control level of the same one or more GAG property is indicative of RA, wherein said control level is comprised within (or comprised within) a reference interval that has been determined for the same sample type obtained from a group of subjects (or from a reference population) not having RA.

In some embodiments, the group of subjects not having OA may be a group of subjects having RA. In some embodiments, the group of subjects not having RA may be a group of subjects having OA.

In some embodiments of methods of the present invention (e.g, methods of screening or diagnosis or discriminating between),
(a) the level of one or more GAG property in accordance with the invention being higher than a control level (or increased in comparison to a control level) of the same one or more GAG property is indicative of OA, wherein said control level is an upper reference limit of a reference interval that has been determined for the same sample type obtained from a group of subjects (or reference population) having RA; or
(b) the level of one or more GAG property in accordance with the invention being lower than a control level (or decreased in comparison to a control level) of the same one or more GAG property is indicative of RA, wherein said control level is an upper reference limit of a reference interval that has been determined for the same sample type obtained from a group of subjects (or reference population) having RA.

In some embodiments of methods of the present invention (e.g, methods of screening or diagnosis or discriminating between),
(a) the level one or more GAG property in accordance with the invention being lower than a control level (or decreased in comparison to a control level) of the same one or more GAG property is indicative of OA, wherein said control level is an upper reference limit of a reference interval that has been determined for the same sample type obtained from a group of subjects (or reference population) having OA; or
(b) the level of one or more GAG property in accordance with the invention being lower than a control level (or decreased in comparison to a control level) of the same one or more GAG property is indicative of RA, wherein said control level is a lower reference limit of a reference interval that has been determined for the same sample type obtained from a group of subjects (or reference population) having OA.

As described above, upper reference limits and lower reference limits may in some cases be used as, or in some cases may be alternatively viewed as, cut-off levels or threshold levels, with an indication of OA or RA being reached (or provided) based on the measured level of one or GAG property in accordance with the invention relative to (or as compared with) such a cut-off limit.

In some preferred embodiments, reference intervals for a particular measurement or value (e.g. the level of one or more of the GAG properties in accordance with the invention) define an interval between which (or a range within which) a 95% of measurements (or values) in a reference population fall into.

In certain alternative embodiments, a reference (or control) range could be generated based on 95% confidence intervals (e.g. 95% confidence around a mean measurement in a group of OA or RA subjects). For example in this regard, and with reference to the experimental Example herein, a reference range for the absolute concentration of 4S CS for a reference population of RA subjects could be 0.939 to 6.573 μg/ml in urine. In some embodiments, an absolute concentration of 4S CS of above 6.573 μg/ml (e.g. in urine) could be indicative of OA. In some embodiments, an absolute concentration of 4S CS of below 6.573 μg/ml (e.g. in urine) could be indicative of RA. Other examples of reference ranges could be derived from data in Table A herein. For the avoidance of doubt, such reference ranges based on Table A are purely exemplary, and the methods of the present invention are not limited thereto.

In some embodiments of methods of the present invention (e.g, methods of screening or diagnosis or discriminating between),
(a) the level of one or more GAG property in accordance with the invention being higher than a control level of the same one or more GAG property is indicative of OA, wherein said control level has been determined in the same sample type obtained from one or more subjects (e.g. group or population of subjects) having RA; or
(b) the level of one or more GAG property in accordance with the invention being lower than a control level of the same one or more GAG property is indicative of RA, wherein said control level has been determined in the same sample type obtained from one or more subjects (e.g. group or population of subjects) having OA.

In some embodiments of methods of the present invention (e.g, methods of screening or diagnosis or discriminating between),
(a) the level of one or more GAG property in accordance with the invention being higher than a control level of the same one or more GAG property is indicative of OA, wherein said control level is the mean or median level that has been determined in the same sample type obtained from a population of subjects having RA; or
(b) the level of one or more GAG property in accordance with the invention being lower than a control level of the same one or more GAG property is indicative of RA, wherein said control level is the mean or median level that has been determined in the same sample type obtained from a population of subjects having OA.

In some embodiments of methods of the present invention (e.g, methods of screening or diagnosis or discriminating between),
(a) the level of one or more GAG property in accordance with the invention being higher than a control level of the same one or more GAG property is indicative of OA, wherein said control level is the level that is at least 20% (or at least 30%, at least 40%, at least 50%, at least 100% or at least 200%) higher than the mean or median level that has been determined in the same sample type obtained from a population of subjects having RA; or
(b) the level of one or more GAG property in accordance with the invention being lower than a control level of the same one or more GAG property is indicative of RA, wherein said control level is the level that is at least 20% (or at least 30%, at least 40%, at least 50%, at least 60%, at least 70%, at least 80%, at least 90% or at least 95%) lower than the mean or median level that has been determined in the same sample type obtained from a population of subjects having OA.

In some embodiments of methods of the present invention (e.g, methods of screening or diagnosis or discriminating between),
(a) the level of one or more GAG property in accordance with the invention being higher than a control level of the same one or more GAG property is indicative of OA, wherein said control level is a level that has been determined by determining the level of said one or more GAG property in the same sample type obtained from one or more subjects (e.g. a population of subjects) having RA and from one or more subjects (e.g. a population of subjects) having OA, wherein the control level is a cut-off level that has been derived (or established or determined) to distinguish (or discriminate) between samples from OA subjects as opposed to samples from RA subjects; or
(b) the level of one or more GAG property in accordance with the invention being lower than a control level of the same one or more GAG property is indicative of RA, wherein said control level is a level that has been determined by determining the level of said one or more GAG property in the same sample type obtained from one or more subjects (e.g. a population of subjects) having RA and from one or more subjects (e.g. a population of subjects) having OA, wherein the control level is a cut-off level that has been derived (or established or determined) to distinguish (or discriminate) between samples from subjects having OA as opposed to samples from subjects having RA.

In some embodiments of methods of the present invention (e.g, methods of screening or diagnosis or discriminating between),
(a) the level of one or more GAG property in accordance with the invention being altered in comparison to (preferably being higher than) a control level of the same one or more GAG property is indicative of OA, wherein said control level is a level that has been determined by determining the level of said one or more GAG property in the same sample type obtained from one or more (e.g. a population of) reference subjects (e.g. reference subjects having RA), wherein the control level is a cut-off level (or threshold level) that has been derived (or established or determined) to provide an indication of OA when the level of the one or more GAG property in accordance with the invention is altered (preferably higher) in comparison thereto; or
(b) the level of one or more GAG property in accordance with the invention being altered in comparison to (preferably being lower than) a control level of the same one or more GAG property is indicative of RA, wherein said control level is a level that has been determined by determining the level of said one or more GAG property in the same sample type obtained from one or more (e.g. a population of) reference subjects (e.g. reference subjects having OA), wherein the control level is a cut-off level that has been derived (or established or determined) to provide an indication of RA when the level of the one or more GAG property in accordance with the invention is altered (preferably lower) in comparison thereto.

In some embodiments of the invention, a control level, e.g. cut-off level (or threshold level), can be derived (or determined or established) to provide a level of confidence in (or for) the indication of OA or RA (as the case may be) in accordance with the invention, for example an (or at least an) 80%, 85%, 90% or 95% level of confidence in (or for) the indication of OA or RA. Thus, in some embodiments of the invention, a control level, e.g. cut-off level (or threshold level), can be derived (or determined or established) to provide a level of confidence in (or for) the indication of OA or RA (as the case may be) in accordance with the invention when an altered level (increased (or higher) or decreased (or lower) as the case may be) of one or more GAG properties in accordance with the invention is determined (or observed) in comparison to a control level (e.g. a cut-off or threshold level). For example, a control level, e.g. cut-off level (or threshold level), can be derived (or determined or established) to provide a level of confidence (e.g. an (or at least an) 80%, 85%, 90% or 95% level of confidence) that an indication (e.g. diagnosis) of OA or RA is accurate (or correct).

In some embodiments of methods of the present invention (e.g, methods of screening or diagnosis or discriminating between),
(a) the level of one or more GAG property in accordance with the invention being within a range (within a range of levels) that has been established as being indicative of the sample being from an OA subject as opposed to being from a RA subject, is indicative of OA; or
(b) the level one or more GAG property in accordance with the invention being within a range (within a range of levels) that has been established as being indicative of the sample being from an RA subject as opposed to being from a OA subject, is indicative of RA.

In some such embodiments, (i) said range of (a) is a range of levels that has been determined by determining the level of said one or more GAG property in the same sample type obtained from a population of subjects having RA and from a population of subjects having OA and establishing a range of levels that is indicative of samples being from subjects having OA as opposed to being from subjects having RA; and/or (ii) said range of (b) is a range of levels that has been determined by determining the level of said one or more GAG property in the same sample type obtained from a population of subjects having RA and from a population of subjects having OA and establishing a range of levels that is indicative of samples being from subjects having RA as opposed to being from subjects having OA.

In some embodiments of methods of the present invention (e.g, methods of screening or diagnosis or discriminating between),
(a) the level of one or more GAG property in accordance with the invention being within a reference interval (or range) that has been established as being (or has been determined to be) indicative of the sample being from an OA subject, is indicative of OA; or
(b) the level of one or more GAG property in accordance with the invention being within a reference interval (or range) that has been established as being (or has been determined to be) indicative of the sample being from an RA subject, is indicative of RA.

In some embodiments of methods of the present invention (e.g, methods of screening or diagnosis or discriminating between),
(a) the level of one or more GAG property in accordance with the invention being a level that has been established as being (or has been determined to be) indicative of the sample being from an OA subject, is indicative of OA; or
(b) the level of one or more GAG property in accordance with the invention being a level that has been established as being (or has been determined to be) indicative of the sample being from an RA subject, is indicative of RA.

In some embodiments of methods of the present invention (e.g, methods of screening or diagnosis or discriminating between),
(a) the level of one or more GAG property in accordance with the invention being a level that has been established as being (or has been determined to be) indicative of the sample being from an OA subject as opposed to from an RA subject, is indicative of OA; or
(b) the level of one or more GAG property in accordance with the invention being a level that has been established as being (or has been determined to be) indicative of the sample being from an RA subject as opposed to from an OA subject, is indicative of RA.

An altered (increased or decreased as the case may be) level (or composition or score) of one or more of the GAG forms (GAG properties) in accordance with the invention includes any measurable alteration or change of the GAG form (biomarker) (or score) in question when the GAG form in question is compared with a control level (e.g. reference limit or cut-off or threshold level). An altered level (or score) includes an increased or decreased level (or score). Preferably, the level (or score) is significantly altered, compared to the level (or score or cut-off level) found in an appropriate control (e.g. control sample or subject or population). More preferably, the significantly altered levels or compositions or scores are statistically significant, preferably with a p-value of <0.05.

In some embodiments, an alteration in level (or score) of ≥2%, ≥3%, ≥5%, ≥10%, ≥25%, ≥50%, ≥75%, ≥100%, ≥200%, ≥300%, ≥400%, ≥500%, ≥600%, ≥700%, ≥800%, ≥900%, ≥1000%, ≥2000%, ≥5000%, or ≥10,000% compared to the level (or score) found in an appropriate control sample or subject or population (i.e. when compared to a control level) may be indicative of the presence of OA or RA (as appropriate) in accordance with the invention.

The “increase” in the level or “increased” level of one or more of the GAG forms (GAG properties) or scores as described herein includes any measurable increase or elevation of the GAG form (biomarker) (or score) in question when the GAG form (or score) in question is compared with a control level (or control score or cut-off level or reference limit). Preferably, the level (or score) is significantly increased, compared to the level (or score or cut-off level) found in an appropriate control (e.g. control sample or subject or population). More preferably, the significantly increased levels (or scores) are statistically significant, preferably with a p-value of <0.05.

In some embodiments, an increase in level (or score) of ≥2%, ≥3%, ≥5%, ≥10%, ≥25%, ≥50%, ≥75%, ≥100%, ≥200%, ≥300%, ≥400%, ≥500%, ≥600%, ≥700%, ≥800%, ≥900%, ≥1000%, ≥2000%, ≥5000%, or ≥10,000% compared to the level (or score) found in an appropriate control sample or subject or population (i.e. when compared to a control level or control score or cut-off level) may be indicative of the presence of OA or RA (as appropriate) in accordance with the invention.

The “decrease” in the level or “decreased” level of one or more of the GAG forms (GAG properties) or scores as described herein includes any measurable decrease or reduction of the GAG form (biomarker) (or score) in question when the GAG form in question is compared with a control level (or control score or cut-off level). Preferably, the level (or score) is significantly decreased, compared to the level (or score or cut-off level) found in an appropriate control (e.g. control sample or subject or population). More preferably, the significantly decreased levels (or scores) are statistically significant, preferably with a p-value of <0.05.

In some embodiments, a decrease in level (or score) of ≥2%, ≥3%, ≥5%, ≥10%, ≥25%, ≥50%, ≥75%, ≥80%, ≥90%, ≥95% or ≥99% compared to the level (or score) found in an appropriate control sample or subject or population (i.e. when compared to a control level or control score or cut-off level) is indicative of the presence of OA or RA (as appropriate) in accordance with the invention.

“Control levels” are discussed elsewhere herein. A “control level” may be the level of a relevant GAG property in a control subject or population (e.g. in a sample(s) that has been obtained from a control subject or population). A “control level” may be a level of a relevant GAG property where the level is derived from (or established based on or calculated from) the level in a control subject or population (e.g. in a sample(s) that has been obtained from a control subject or population). Such a population may be referred to as a reference population. Appropriate control subjects (or populations) or samples for use in methods of the invention would be readily identified by a person skilled in the art. Suitable control levels are described elsewhere herein. The control level may correspond to the level of the equivalent (corresponding) GAG form in appropriate control subjects or samples or populations, e.g. may correspond to a cut-off or threshold level or range found in a control or reference population. Control levels may also be referred to as “reference” levels. The control level may be a discrete figure or a range.

Although the control level for comparison could be derived by testing an appropriate control subject or set of control subjects (or a control population), the methods of the invention would not necessarily involve carrying out active tests on control subjects as part of the methods of the present invention but would generally involve a comparison with a control level which had been determined previously from control subjects (or a control population) and was known to the person carrying out the methods of the invention.

A “control chemical composition” is the chemical composition in a control subject or population (e.g. in a sample that has been obtained from a control subject or population). The discussion above in relation to a “control level” (e.g. appropriate control subjects, control samples, control populations, etc.) may be applied, mutatis mutandis, to “a control chemical composition”.

As described elsewhere herein, screening for OA and RA in accordance with the present invention may involve using a score (or a GAG score), or expressing the level and/or chemical composition determined in accordance with the invention using a score (or GAG score). In some such embodiments, an altered score (e.g. increased or decreased as the case may be) in comparison to a “control score” (or cut-off level or threshold level) is indicative of OA or RA (as appropriate) in said subject. The discussion elsewhere herein in relation to a “control level” (e.g. appropriate control subjects, control samples, control populations, etc.) may be applied, mutatis mutandis, to a “control score”.

As described elsewhere herein, in some preferred methods of the present invention, the method comprises determining the level and/or chemical composition in accordance with the invention of the protein-free fraction of one or both of the GAGs chondroitin sulfate (CS) and heparan sulfate (HS) (or a score based thereon). In some such embodiments, an altered level and/or chemical composition of the protein-free fraction (or a score based thereon) in comparison to a level and/or chemical composition of the protein-free fraction of said one or both GAGs in a control (e.g. control sample or control score or cut-off level) is indicative of OA or RA (as appropriate). The discussion elsewhere herein in relation to a “control level” or “control chemical composition” or “control score” (e.g. appropriate control subjects, control samples, control populations, etc.) may be applied, mutatis mutandis, to embodiments of the invention that comprise determining the level and/or chemical composition in accordance with the invention of the protein-free fraction of one or both of said GAGs (or a score based thereon).

Methods of the present invention can also be used to monitor OA progression (e.g. OA worsening) or RA progression (e.g. RA worsening). Such monitoring can take place before, during or after treatment of OA or RA by surgery or therapy, e.g. pharmaceutical therapy. Thus, in another aspect the present invention provides a method for monitoring the progression of OA or RA in a subject having OA or RA. In such methods for monitoring the progression of OA or RA in a subject, the level of one or more of the GAG properties in accordance with the present invention (or a score derived or based thereon) is indicative of the progression of OA or RA (as appropriate).

Thus, in another aspect, the present invention provides a method of monitoring the progression of osteoarthritis (OA) or rheumatoid arthritis (RA) in a subject having arthritis (OA or RA), said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS and (v) total concentration of HS,
wherein said sample has been obtained from said subject.

In embodiments in which the progression of OA is monitored, the subject is a subject having OA.

In embodiments in which the progression of RA is monitored, the subject is a subject having RA.

In preferred embodiments of methods of monitoring OA or RA progression of the invention, an altered level (increased level or decreased level as the case may be) of one or more of the GAG properties in accordance with the invention (4S CS (e.g. absolute concentration of 4S CS), NS HS (e.g. absolute concentration of NS HS and/or relative concentration of NS HS), 0S HS (e.g. absolute concentration of 0S HS), total concentration of CS and/or total concentration of HS) in comparison to a control level is indicative of OA or RA progression in said subject.

In some embodiments of methods of monitoring progression in accordance with the present invention
(a) progression of OA is monitored and an altered (or different) level, preferably an increased (or higher) level, of one or more GAG property in accordance with the invention over time as compared to a control level of the same one or more GAG property is indicative of progression of OA, wherein said control level has been determined in the same sample type obtained from one or more subjects not having OA or has been determined in the same sample type obtained from an earlier sample taken from the subject whose OA progression is being monitored; or
(b) progression of RA is monitored and an altered (or different) level, preferably a decreased (or lower) level, of one or more GAG property in accordance with the invention over time as compared to a control level of the same one or more GAG property is indicative of progression of RA, wherein said control level has been determined in the same sample type obtained from one or more subjects not having RA or has been determined in the same sample type obtained from an earlier sample taken from the subject whose RA progression is being monitored.

In some embodiments of methods of monitoring progression of OA in accordance with the invention, the control level may be a level of one or more GAG property in accordance with the invention determined in the same sample type obtained from one or more subjects (e.g. a population (e.g. a reference population) of subjects) not having OA.

In some embodiments of methods of monitoring progression of RA in accordance with the invention, the control level may be a level of one or more GAG property in accordance with the invention determined in the same sample type obtained from one or more subjects (e.g. a population (e.g. a reference population) of subjects) not having RA.

In some preferred embodiments of methods of monitoring progression in accordance with the present invention,
(a) progression of OA is monitored and an increasing level of one or more GAG property in accordance with the invention over time as compared to a control level of the same one or more GAG property is indicative of progression of OA, wherein said control level has been determined in the same sample type obtained from an earlier sample taken from the subject whose OA progression is being monitored; or
(b) progression of RA is monitored and a decreasing level of one or more GAG property in accordance with the invention over time as compared to a control level of the same one or more GAG property is indicative of progression of RA, wherein said control level has been determined in the same sample type obtained from an earlier sample taken from the subject whose RA progression is being monitored.

Thus, in some embodiments of methods of monitoring progression of OA in accordance with the invention, the control level is a level that has been determined in the same sample type obtained from an earlier (e.g. a first) sample taken from the subject whose OA progression is being monitored.

In some embodiments of methods of monitoring progression of RA in accordance with the invention, the control level is a level that has been determined in the same sample type obtained from an earlier (e.g. a first) sample taken from the subject whose RA progression is being monitored.

A level from an earlier (e.g. first) sample from the subject whose OA or RA progression (as appropriate) is being monitored may be considered a “baseline” level in the subject. This type of control level (i.e. a control level from an individual subject) is particularly useful for embodiments of the invention where serial or periodic measurements of GAG form(s) in individuals are taken looking for changes in the levels of the GAG form(s). In this regard, an appropriate control level can be the individual's own baseline, stable, nil, previous or dry value (as appropriate) as opposed to a control or cut-off level found in the general control population. Thus, a control level for methods of monitoring progression of OA or RA (as appropriate) in accordance with the invention may correspond to the level of the marker (GAG form) in question in the same individual subject, or a sample from said subject, measured at an earlier time point (e.g. a “baseline” level in that subject).

In some embodiments, the level of one or more of the GAG properties in accordance with the invention is indicative of OA progression, preferably with high (or higher) or increased (or increasing) levels (e.g. as measured over time e.g. by taking serial or periodical measurements) being indicative of OA progression (OA worsening).

In some embodiments, the level of one or more of the GAG properties in accordance with the invention is indicative of RA progression, preferably with low (or lower) or decreased (or decreasing) levels (e.g. as measured over time e.g. by taking serial or periodical measurements) being indicative of RA progression (RA worsening).

In preferred methods of monitoring progression in accordance with the invention, the body fluid sample is a urine sample (or a processed urine sample).

Preferred GAG properties (or groups of GAG properties) for use in connection with methods of monitoring progression in accordance with the invention are discussed elsewhere herein in connection with other aspects of the invention. For example, 4S CS and NS HS are typically preferred.

Sample processing step and/or detection (or measurement methods, etc.) for use in connection with methods of monitoring progression in accordance with the invention are discussed elsewhere herein in connection with other aspects of the invention. Indeed, unless otherwise clear from the context, features described elsewhere herein in connection with methods of screening (e.g. diagnosis etc.) may also be applied to methods of monitoring progression of the invention.

In a further aspect, the present invention provides a method of providing information that is useful for monitoring progression of osteoarthritis (OA) or rheumatoid arthritis (RA) in a subject having arthritis, said method comprising:
determining the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject. Said information is of course typically the determined level of one or more of said GAG properties.

Methods of the present invention can be used in the active monitoring of patients which have not been subjected to surgery or therapy, e.g. to monitor the progression of OA or RA (as appropriate) in untreated patients. Again, serial measurements can allow an assessment of whether or not, or the extent to which, the OA or RA (as appropriate) is worsening, thus, for example, allowing a more reasoned decision to be made as to whether therapeutic or surgical intervention is necessary or advisable.

In another aspect, the present invention provides a method for determining the clinical severity of OA or RA (as appropriate) in a subject. In such methods the level of one or more of the GAG forms in accordance with the invention in the sample (or a score derived therefrom or based thereon) shows an association with the severity of the OA or RA (as appropriate). Thus, the level of one or more of the GAG forms in accordance with the present invention can be indicative of the severity of the OA or RA. In some embodiments, the more altered (more increased or more decreased as the case may be) the level of one or more of the GAG forms in accordance with the invention in comparison to a control level (e.g. a control level as described elsewhere herein), the greater the likelihood of a more severe form of OA or RA (as appropriate). In some embodiments, the methods of the invention can thus be used in the selection of patients for therapy.

Serial (periodical) measuring of the level of one or more of the GAG forms in accordance with the invention (biomarkers) (or a score derived therefrom or computed based thereon) may also be used to monitor the severity of OA or RA (as appropriate) looking for either increasing or decreasing levels over time. Observation of altered levels (increase or decrease as the case may be) may also be used to guide and monitor therapy (or other clinical decisions), for example in the situation of “watchful waiting” before treatment or surgery, e.g. before initiation of pharmaceutical therapy or surgery, or during or after treatment to evaluate the effect of treatment and look for signs of therapy failure.

The present invention also provides a method of patient selection or treatment selection as it provides a means of distinguishing (or discriminating between) patients with OA from patients with RA. Thus, alternatively viewed, methods of the present invention provide a method for distinguishing (or discriminating between) OA and RA. Such methods may guide appropriate treatment.

The present invention also provides a method of patient selection or treatment selection as it provides a means of distinguishing (or discriminating between) patients with severe OA from patients with less severe OA (e.g. identifying, or providing an indication of, patients with a particular clinical stage of OA, e.g. stage 1, stage 2, stage 3 or stage 4 OA).

The present invention also provides a method of patient selection or treatment selection as it provides a means of distinguishing (or discriminating between) patients with severe RA from patients with less severe RA (e.g. identifying, or providing an indication of, patients with a particular clinical stage of RA, e.g. stage 1, stage 2, stage 3 or stage 4 RA). The classification of OA or RA at a given stage may be in accordance with any art recognised and accepted definition. The skilled person is familiar with staging systems and conventions OA and RA.

In some embodiments, the invention provides a method of monitoring (e.g. continuously monitoring or performing active surveillance of) a subject having OA or RA (e.g. a subject being treated for OA or RA). Such monitoring may guide which treatment to use or whether no treatment should be given.

In some embodiments, patients with less severe (or early stage) OA or RA may be put under watchful waiting or active surveillance and may not be given treatment (e.g. pharmaceutical therapy or surgery). In some embodiments, patients with severe or more severe (or late stage) OA or RA may be given treatment.

The present invention also provides a method of determining (or monitoring) the efficacy of a therapeutic regime being used to treat OA or RA (as appropriate), in other words following or monitoring a response to treatment. In such methods, an alteration (increase or decrease as the case may be) in the level (or scores) of one or more of the GAG properties in accordance with the present invention indicates the efficacy of the therapeutic regime being used. For example, in some embodiments, if the level of one or more of the GAG forms in accordance with the present invention (or a score derived therefrom (or based thereon)) for which an increased level (or score) is indicative of OA is reduced during (or after) therapy, this is indicative of an effective therapeutic regime. By way of another example, in some embodiments, if the level of one or more of the GAG forms in accordance with the present invention (or a score derived therefrom (or based thereon)) for which a decreased level (or score) is indicative of RA is increased during (or after) therapy, this is indicative of an effective therapeutic regime. In such methods, serial (periodical) measuring of the level of one or more of the GAG properties (biomarkers) in accordance with the invention over time can also be used to determine the efficacy of a therapeutic regime being used. Similar methods can be used to provide a method of determining (or monitoring) the efficacy of a surgical regime being used to treat OA or RA.

The methods of the present invention can be carried out on any appropriate body fluid sample. Typically the sample has been obtained from (removed from) a subject (e.g. as described elsewhere herein), preferably a human subject. In other aspects, the method further comprises a step of obtaining a sample from the subject.

Reference herein to “body fluid” includes reference to all fluids derived from the body of a subject. The body fluid or sample may be in the form of a liquid biopsy. A preferred body fluid in accordance with the invention is urine. Urine is a particularly preferred body fluid as it is an easily accessible body fluid. Thus, in preferred embodiments, the level of a GAG property in accordance with the invention is the level in a urine sample.

The term “sample” also encompasses any material derived by processing a body fluid sample (e.g. derived by processing a urine sample). Thus the term “sample” includes a processed sample (e.g. a processed urine sample). Processing of biological samples to obtain a test sample may involve one or more of: digestion, boiling, filtration, distillation, centrifugation, lyophilization, fractionation, extraction, concentration, dilution, purification, inactivation of interfering components, addition of reagents, derivatization, complexation and the like, e.g. as described elsewhere herein. Suitable processing steps can be selected depending on the features of the method being performed.

Any suitable method for isolating body fluid samples (e.g. urine samples) may be employed.

Any sample that could be directly or indirectly affected by OA or RA may be used. Samples (e.g. original or unprocessed samples) typically comprise a protein-free fraction of GAGs and a protein-bound fraction of GAGs (as discussed elsewhere herein). In preferred methods of the present invention, the level and/or chemical composition of the protein-free fraction of one or both of the GAGs chondroitin sulfate (CS) and heparan sulfate (HS) is determined. Thus, in certain preferred embodiments a body fluid sample has been processed (or is processed) such that only (or essentially only) the protein-free fraction of said GAGs (typically disaccharide units derived therefrom) is subsequently analysed (i.e. processed such that the level and/or chemical composition of the protein-free fraction of said one or both GAGs (typically disaccharide units derived therefrom) is subsequently determined). In other methods of the invention, the sample has been processed (or is processed) such that the entire (protein-free plus protein-bound) fraction or pool of said one or both GAGs is subsequently analysed (i.e. processed such that the level and/or chemical composition of the entire (protein-free plus protein-bound) fraction or pool of said one or both GAGs (typically disaccharide units derived therefrom) is subsequently determined).

The term “sample” also encompasses any material derived by processing (e.g. as described above) a biological sample. Derived materials include disaccharide units (or a population of disaccharide units) derived by processing GAGs (e.g. as described elsewhere herein).

In some embodiments, methods of the invention may include a step of processing a sample. In some embodiments, methods of the invention may thus be performed on such processed samples or materials derived from such processed samples. In some embodiments, methods of the invention may thus be performed on samples that have been processed. Processing steps include, but are not limited to, isolating cells from the sample, isolating cell components from the sample, and extracting (e.g. isolating or purifying) proteins/peptides (although as certain preferred methods of the invention involve the determination of the protein-free GAG fraction, the extraction of proteins or the removal of the protein component from the proteoglycans (protein-bound GAGs) present in the sample, e.g. by digesting or otherwise removing the protein component, is preferably not carried out in some embodiments). A processing step may involve one or more of filtration, distillation, centrifugation, extraction, concentration, dilution, purification, inactivation of interfering components, addition of reagents, derivatization, amplification, adapter ligation, and the like.

Samples can be used immediately or can be stored for later use (e.g. at −80° C.).

The methods of the invention as described herein can be carried out on any type of subject which is capable of suffering from OA or RA. The methods are generally carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs). Preferably the subject is a human.

In some embodiments of the present invention, the subject (e.g. a human) is a subject having arthritis. Thus, in some embodiments, the subject is a subject that has been diagnosed with arthritis (e.g. OA or RA). In other embodiments, the subject (e.g. a human) is a subject suspected of having arthritis (e.g. OA or RA). In methods of monitoring progression in accordance with the invention the subject is a subject having arthritis (having OA or having RA).

In some aspects, methods of the invention are provided which further comprise a step of treating OA or RA (as appropriate) by therapy (e.g. pharmaceutical therapy), or surgery, or other clinical management program. Methods of treating OA and RA by therapy or surgery or by prescribing another clinical management program are known in the art.

In some embodiments, if the result of a method of the invention is indicative of OA (or indicative of OA progression (worsening)) in the subject (e.g. if a positive indication or diagnosis of OA is made), then an additional step of treating the OA by therapy or surgery or providing a clinical management program appropriate for OA can be performed. For example, if the result of a method of the invention is indicative of OA (or indicative of OA progression (worsening)) in the subject, the subject may be instructed (or advised) to make certain lifestyle changes (e.g. to exercise or more regularly exercise), the subject may be instructed (or advised) to lose weight (e.g. if a subject is overweight), the subject may be instructed (or advised) to wear suitable (or more suitable) footwear and/or the subject may be instructed (or advised) to use a device to reduce the strain on joints. By way of a further example, if the result of a method of the invention is indicative of OA (or indicative of OA progression (worsening)) in the subject, the subject may be alternatively, or additionally, prescribed a physical therapy program and/or the subject may be prescribed painkillers and/or the subject may have joint replacement surgery.

In some embodiments, if the result of a method of the invention is indicative of RA (or indicative of RA progression (worsening)) in the subject (e.g. a positive indication or diagnosis of RA is made), then an additional step of treating the RA by therapy or surgery or providing a clinical management program appropriate for RA can be performed. For example, if the result of a method of the invention is indicative of RA (or indicative of RA progression (worsening)) in the subject, the subject may be treated with (or prescribed) a disease-modifying anti-rheumatic drug (DMARD) (such as methotrexate, leflunomide, hydroxychloroquine, or sulfasalazine) and/or a JAK inhibitor and/or painkillers and/or steroids and/or non-steroidal anti-inflammatory drugs (NSAIDS) and/or a biological agent (such as adalimumab, etanercept or infliximab). By way of an further example, if the result of a method of the invention is indicative of RA (or indicative of RA progression (worsening)) in the subject, the subject may be alternatively, or additionally, prescribed a physical therapy program and/or the subject may have joint replacement surgery.

In some embodiments, if the level of one or more GAG properties in accordance with the invention in a sample (or a score based on these levels) is altered by a particular degree in comparison to a control level or score (or cut-off level), then a further step of administering a therapeutically effective amount of a pharmaceutical agent to the patient is performed and/or surgery is performed. Preferred degrees of alteration are discussed elsewhere herein.

In some aspects, methods of the invention are provided which further comprise a step of carrying out (or performing) an additional diagnostic or screening procedure for OA or RA (as appropriate), e.g. as discussed elsewhere.

A yet further aspect provides a kit for screening for OA or RA (e.g. for diagnosing or for determining severity or progression of OA or RA or for discriminating between OA and RA), which comprises one or more agents suitable for determining the level of one or more of the GAG properties (GAG forms) described herein in accordance with the invention, in a sample. A yet further aspect provides a kit for screening for OA or RA (e.g. for diagnosing or for determining severity or progression of Oa or RA), which comprises one or more reagents (or components) for processing a body fluid sample (e.g. urine) that comprises GAGs whose level and/or chemical composition is determined in accordance with the invention. In preferred aspects said kits are for use in the methods of the invention as described herein. Preferably said kits comprise instructions for use of the kit components, for example in screening (e.g. diagnosis) in accordance with the invention.

In another aspect, the present invention provides a method of determining (or detecting) the level and/or chemical composition of one or both of the glycosaminoglycans (GAGs) chondroitin sulfate (CS) and heparan sulfate (HS) in a body fluid sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS and (v) total concentration of HS,
wherein said sample has been obtained from a subject having, or suspected of having, OA or RA.

In one aspect, the present invention provides a method of detecting (or determining) the level and/or chemical composition of one or both of the glycosaminoglycans (GAGs) chondroitin sulfate (CS) and heparan sulfate (HS), said method comprising:
(a) obtaining a body fluid sample from a human patient having, or suspected of having, OA or RA; and
(b) detecting (or determining) the level and/or chemical composition of one or both of the glycosaminoglycans (GAGs) chondroitin sulfate (CS) and heparan sulfate (HS) in said sample,
wherein said detecting (or determining) the level and/or chemical composition of one or both of said GAGs comprises determining the level of one or more GAG property selected from the group consisting of: (i) 4S CS, (ii) NS HS, (iii) 0S HS, (iv) total concentration of CS and (v) total concentration of HS.

A yet further aspect of the invention provides a method of detecting (or determining) the level and/or chemical composition of one or both of the glycosaminoglycans (GAGs) chondroitin sulfate (CS) and heparan sulfate (HS) in a body fluid sample,
wherein said detecting (or determining) the level and/or chemical composition of one or both of said GAGs comprises determining the level of one or more GAG property selected from the group consisting of: (i) 4S CS, (ii) NS HS, (iii) 0S HS, (iv) total concentration of CS and (v) total concentration of HS,
wherein said sample has been obtained from said subject having OA or RA, or
suspected of having OA or RA, and has been subjected to processing prior to
determining said level and/or chemical composition, wherein said processing
(a) comprises fragmenting said one or both GAGs into
   disaccharide units; and
(b) does not comprise prior to (a) at least one of:
   (i) contacting said sample with a proteolytic agent; and
   (ii) purifying said one or both GAGs in said sample based on the negative charge of said GAGs.

The features and discussion herein in relation to the method of screening for OA or RA (e.g. method of diagnosing, etc.), for example in relation to preferred GAG forms or combinations thereof for measurement, can be applied, mutatis mutandis, to methods of detecting of the present invention.

Where the terms “comprise”, “comprises”, “comprising”, “has” or “having”, or other equivalent terms are used herein, then in some more specific embodiments these terms include the term “consists of” or “consists essentially of”, or other equivalent terms.

The invention will be further described with reference to the following non-limiting Example:

### EXAMPLE

### Introduction

We investigated urine glycosaminoglycans (GAGs) profiles—or GAGomes—comprising chondroitin sulfate (CS) and heparan sulfate (HS) disaccharides as biomarkers of osteoarthritis (OA) and rheumatoid arthritis (RA). In this study, the GAGome of the protein-free fraction of GAGs (or free GAGome) of urine samples was studied.

### Patients and Methods

We conducted a prospective single-center case-control study. The study population is representative of patients with OA or RA. The inclusion criteria were:

for the OA arm: diagnosis of osteoarthritis (knee pain for >6 months and radiology-confirmed diagnosis); age≥18 years old;

for the RA arm: diagnosis of rheumatoid arthritis (as confirmed by fulfilling the American Rheumatism Association criteria for RA); age≥18 years old.

No patients were excluded. All patients signed the informed consent form. Any void spot urine samples from eligible patients were obtained in a single visit on a single collection cup, which was successively stored at −20° C. until analysis. Patients were assigned to two groups depending on the inclusion arm, OA vs. RA. Patient characteristics are summarized in Table 1.

TABLE 1
Study population characteristics.

| | OA | RA | p |
|---|---|---|---|
| n | 10 | 10 | |
| Gender = Male (%) | 3 (30.0) | 2 (20.0) | 1.000 |
| Age (mean (SD)) | 68.80 (7.07) | 46.00 (15.18) | <0.001 |

We measured the urine GAGome using a standardized UHPLC-MS/MS method (D. Tamburro, S. Bratulic, S. A. Shameh, N. K. Soni, A. Bacconi, F. Maccari, F. Galeotti, K. Mattsson, N. Volpi, J. Nielsen, F. Gatto, Journal of Chromatography B, 1177 (2021) 122761) in a single blinded laboratory. Briefly, GAGome extraction from each sample was performed following Elypta MIRAM® Free Glycosaminoglycan Kit instructions for use. All reagents and consumables used were contained in the kit. The method for GAGome extraction included an enzymatic digestion assay using Chondroitinase ABC and Heparinase I-II-III to depolymerize GAGs in the sample into disaccharides. Note that compared to other methods described in the art (see Volpi et al., Nature Protocols, 9, 541-558 (2014)) where use of a non-specific protease is recommended for biological fluid analysis, the present method omitted the addition of a protease and thus the analysis was limited to the protein-free fraction of GAGs. Note that as compared to Volpi et al., the method used in the present study omitted the step of purifying the GAGs using an anion-exchange resin.

GAG disaccharides were subsequently labeled using 2-aminoacridone. The processed samples were then injected into an ultra-high-performance liquid chromatography (UHPLC) coupled with electrospray ionization triple-quadrupole mass spectrometry system (ESI-MS/MS, Waters® 6 Acquity I-class Plus Xevo TQ-S micro) for disaccharide separation and detection. The peaks of GAG disaccharides (listed below) were acquired at pre-specified retention times across six transitions using multiple reaction monitoring (MRM) analysis implemented in the mass spectrometry software (Waters® 9 TargetLynx). We used the mass spectrometry software (Waters® TargetLynx) for peak integration, construction of calibration curves, and quantification.

We exported the results processed data in Excel format and imported it into R (4.0.2) for comparing the level of each free GAGome feature between OA and RA. A two-sided t-test was used to assess a difference between groups. A p-value<0.05 was considered statistically significant.

The measured GAG profiles (GAGomes) consisted of absolute concentrations for 17 GAG disaccharides, corresponding to 8 different sulfation patterns of chondroitin sulfate (CS) and heparan sulfate (HS), and hyaluronic acid (HA) disaccharide. Specifically, we quantified 8 CS disaccharides (0S CS, 2S CS, 6S CS, 4S CS, 2S6S CS, 2S4S CS, 4S6S CS, Tris CS) and 8 HS disaccharides (0S HS, 2S HS, 6S HS, NS HS, NS6S HS, NS2S HS, 2S6S HS, Tris HS). The GAGome was expanded to include an additional 22 dependent features: the total CS and total HS concentration as the sum of the corresponding disaccharide concentrations, the CS and HS charge, two ratios (4S CS/0S CS and 6S CS/0S CS), and the relative concentration (or mass fraction, in %) of each of the 16 CS and HS disaccharide by normalizing its absolute concentration by the total CS and HS concentration, respectively. For a urine sample, the GAGome, therefore, consisted of 39 GAG features.

### Results

The free GAGome features listed in Table A (the features with the suffix [μg/ml] in Table A) were detected in most patients in the study (>0.1 μg/mL in at least 10 patients out of the total of 20 patients in the study). Specifically, we detected six CS disaccharides (0S CS, 6S CS, 4S CS, 2S6S CS, 2S4S CS, 4S6S CS) at a concentration of >0.1 μg/ml in samples obtained from at least 10 patients out of the total of 20 patients in the study, and two HS disaccharides (0S HS, NS HS) at a concentration of >0.1 μg/ml in samples obtained from at least 10 patients out of the total of 20 patients in the study. The free GAGome was also expanded to include additional dependent features: the total CS concentration as the sum of the 8 CS disaccharide concentrations mentioned above and total HS concentration as the sum of the 8 HS disaccharide concentrations mentioned above, the CS and HS charge, two ratios (4S CS/0S CS and 6S CS/0S CS), and the relative concentration (or mass fraction, in %) of each of the six CS disaccharides (0S CS, 6S CS, 4S CS, 2S6S CS, 2S4S CS, 4S6S CS) and two HS disaccharides (0S HS, NS HS) mentioned above by normalizing its absolute concentration by the total CS and HS concentration, respectively.

TABLE A

| | | | | | Mean | Std. | Lower | Upper | Mean | Std. | Lower | Upper | | |
|---|---|---|---|---|---|---|---|---|---|---|---|---|---|---|
| | | | | | in | dev. | Lim 95% | Lim 95% | in | dev. | Lim 95% | Lim 95% | | |
| GAGome feature | | | n1 | n2 | OA | in OA | CI for OA | CI OA | RA | in RA | CI RA | CI RA | df | p |
| 4S CS urine [ug/mL] | OA | RA | 10 | 10 | 6.343 | 3.101 | 0.266 | 12.420 | 3.756 | 1.437 | 0.939 | 6.573 | 12.697 | 0.033 |
| NS HS urine [ug/mL] | OA | RA | 10 | 10 | 0.712 | 0.542 | 0.000 | 1.774 | 0.290 | 0.203 | 0.000 | 0.688 | 11.483 | 0.041 |
| OS HS urine [ug/mL] | OA | RA | 10 | 10 | 2.685 | 2.178 | 0.000 | 6.955 | 1.262 | 0.817 | 0.000 | 2.864 | 11.485 | 0.078 |
| Total CS urine | OA | RA | 10 | 10 | 12.249 | 6.061 | 0.370 | 24.127 | 8.103 | 3.687 | 0.877 | 15.329 | 14.858 | 0.085 |
| NS HS urine | OA | RA | 10 | 10 | 21.574 | 8.859 | 4.210 | 38.938 | 15.321 | 6.415 | 2.747 | 27.895 | 16.403 | 0.089 |
| Total HS urine | OA | RA | 10 | 10 | 3.771 | 2.833 | 0.000 | 9.324 | 1.943 | 1.479 | 0.000 | 4.843 | 13.569 | 0.093 |
| 2S4S CS urine [ug/mL] | OA | RA | 10 | 10 | 0.153 | 0.082 | 0.000 | 0.314 | 0.100 | 0.064 | 0.000 | 0.226 | 17.039 | 0.123 |
| 4S CS urine | OA | RA | 10 | 10 | 51.465 | 5.717 | 40.260 | 62.670 | 47.856 | 5.444 | 37.185 | 58.527 | 17.957 | 0.166 |
| 6S CS urine | OA | RA | 10 | 10 | 22.001 | 8.018 | 6.285 | 37.717 | 26.012 | 5.038 | 16.137 | 35.887 | 15.148 | 0.200 |
| 0S CS urine [ug/mL] | OA | RA | 10 | 10 | 2.639 | 2.366 | 0.000 | 7.277 | 1.577 | 0.883 | 0.000 | 3.308 | 11.460 | 0.210 |
| 4S6S CS urine [ug/mL] | OA | RA | 10 | 10 | 0.356 | 0.215 | 0.000 | 0.777 | 0.270 | 0.156 | 0.000 | 0.575 | 16.430 | 0.316 |
| 2S6S CS urine [ug/mL] | OA | RA | 10 | 10 | 0.258 | 0.211 | 0.000 | 0.672 | 0.181 | 0.118 | 0.000 | 0.412 | 14.152 | 0.328 |
| 4S6S CS urine | OA | RA | 10 | 10 | 2.630 | 1.222 | 0.235 | 5.026 | 3.129 | 1.735 | 0.000 | 6.530 | 16.166 | 0.468 |
| 6S CS urine [ug/mL] | OA | RA | 10 | 10 | 2.489 | 1.296 | 0.000 | 5.029 | 2.214 | 1.191 | 0.000 | 4.549 | 17.873 | 0.627 |
| 2S4S CS urine | OA | RA | 10 | 10 | 1.194 | 0.427 | 0.357 | 2.031 | 1.101 | 0.493 | 0.134 | 2.068 | 17.638 | 0.657 |
| 2S6S CS urine | OA | RA | 10 | 10 | 2.092 | 1.21 | 0.000 | 4.472 | 1.965 | 0.963 | 0.077 | 3.852 | 17.114 | 0.798 |
| 4S/0S CS urine | OA | RA | 10 | 10 | 3.017 | 2.795 | 0.000 | 8.494 | 2.767 | 1.413 | 0.000 | 5.536 | 13.319 | 0.804 |
| Charge CS urine | OA | RA | 10 | 10 | 0.804 | 0.131 | 0.548 | 1.060 | 0.814 | 0.067 | 0.683 | 0.944 | 13.381 | 0.841 |
| 0S CS urine | OA | RA | 10 | 10 | 20.552 | 13.506 | 0.000 | 47.023 | 19.797 | 6.758 | 6.551 | 33.043 | 13.241 | 0.877 |
| Charge HS urine | OA | RA | 10 | 10 | 0.369 | 0.163 | 0.050 | 0.688 | 0.376 | 0.256 | 0.000 | 0.878 | 15.239 | 0.945 |
| 6S/0S CS urine | OA | RA | 10 | 10 | 1.487 | 1.870 | 0.000 | 5.152 | 1.503 | 0.702 | 0.128 | 2.878 | 11.485 | 0.980 |
| 0S HS urine | OA | RA | 10 | 10 | 67.759 | 11.645 | 44.934 | 90.584 | 67.672 | 21.021 | 26.471 | 108.872 | 14.049 | 0.991 |

In the “GAGome feature” column of Table A above, the rows with [ug/mL] after the stated GAGome feature show the absolute concentration of the stated GAGome feature (microgram/mL; μg/ml). Total CS and Total HS concentrations are also in μg/ml. Charge CS and Charge HS are discussed elsewhere herein. The ratios (4S/0S CS and 6S/0S CS) are discussed elsewhere herein. The other rows for the stated sulfated or unsulfated GAGome features that do not have a [ug/mL] suffix show the relative concentration of the stated GAGome feature (microgram/microgram total for the relevant GAG group, e.g. CS) (also viewed as “mass fraction” - relative concentration is also explained elsewhere herein).
“CI” = confidence interval.

We observed a statistically significant difference in the level of 4S CS (μg/ml) and NS HS (μg/ml) (p<0.05), wherein samples from OA patients displayed ˜2× higher concentration of these disaccharides compared to RA patients. The data also points to other differences between OA and RA (p<0.1), including an increase of 0S HS (μg/ml), total concentration of CS and total concentration of HS in OA vs. RA. Although the sample size was likely underpowered to meet statistical significance in the case of the observed increases in 0S HS (μg/ml), total concentration of CS and total concentration of HS in OA vs. RA, a clear trend of increases in these GAGome features was observed.

In conclusion, this data clearly supports the notion that OA and RA display different urine GAGome profiles, with several disaccharides having different concentrations between the groups (i.e. between OA and RA). These results indicate that urine GAGome profiles in accordance with the present invention could be used as diagnostic or screening biomarkers for OA or RA. In addition, the biomarkers in accordance with the present invention could be used to differentiate between OA and RA by, for example, establishing reference intervals around one or more disaccharides here identified as different in OA vs. RA.

1. A method of discriminating between osteoarthritis (OA) and rheumatoid arthritis (RA) in a subject having arthritis or in a subject suspected of having arthritis, said method comprising:
determining the level and/or chemical composition of one or both of the glycosaminoglycans (GAGs) chondroitin sulfate (CS) and heparan sulfate (HS) in a urine sample,
wherein said determining the level and/or chemical composition of one or both of said GAGs comprises determining the level of one or more GAG property selected from the group consisting of: (i) 4S CS, (ii) NS HS, (iii) 0S HS, (iv) total concentration of CS, and (v) total concentration of HS,
wherein said sample has been obtained from said subject.

2. The method of claim 1, wherein
(a) the level of said one or more GAG property being higher than a control level of the same one or more GAG property is indicative of OA, wherein said control level has been determined in the same sample type obtained from one or more subjects having RA; or
(b) the level of said one or more GAG property being lower than a control level of the same one or more GAG property is indicative of RA, wherein said control level has been determined in the same sample type obtained from one or more subjects having OA.

3. The method of claim 1, wherein
(a) the level of said one or more GAG property being different than a control level of the same one or more GAG property is indicative of OA, wherein said control level is comprised within a reference interval that has been determined for the same sample type obtained from a group of subjects not having OA; or
(b) the level of said one or more GAG property being different than a control level of the same one or more GAG property is indicative of RA, wherein said control level is comprised within a reference interval that has been determined for the same sample type obtained from a group of subjects not having RA.

4. The method of any one of claims 1 to 3, wherein said determining the level and/or chemical composition of one or both of said GAGs comprises determining the level of one or both of the GAG properties: 4S CS and NS HS.

5. The method of any one of claims 1 to 4, wherein said determining the level and/or chemical composition of one or both of said GAGs comprises determining the level of one or more GAG property selected from the group consisting of:
(i) absolute concentration of 4S CS,
(ii) absolute concentration of NS HS and/or relative concentration of NS HS,
(iii) absolute concentration of 0S HS,
(iv) total concentration of CS; and
(v) total concentration of HS.

6. The method of any one of claims 1 to 5, wherein said determining the level and/or chemical composition of one or both of said GAGs comprises determining the level of one or both GAG properties selected from the group consisting of absolute concentration of 4S CS and absolute concentration of NS HS.

7. The method of any one of claims 1 to 6, wherein said method comprises determining the level of more than one of said GAG properties, preferably said method comprises determining the level of two or more, three or more, four or more, or all, of said GAG properties.

8. The method of any one of claims 1 to 7, wherein said determining the level and/or chemical composition is determining the level and/or chemical composition of the protein-free fraction of one or both of the glycosaminoglycans (GAGs) chondroitin sulfate (CS) and heparan sulfate (HS).

9. The method of any one of claims 1 to 8, wherein said sample has been obtained from said subject and has been subjected to at least one processing step prior to determining said level and/or chemical composition.

10. The method of any one of claims 1 to 9, wherein the levels of one or more specific sulfated or unsulfated forms of CS or HS disaccharides are determined, and wherein the GAGs are, or have been, subjected to a processing step to obtain disaccharide units for analysis.

11. The method of claim 9 or claim 10, wherein said at least one processing step does not comprise contacting said sample with a proteolytic agent.

12. The method of any one of claims 1 to 11, wherein said sample has been obtained from said subject and has been subjected to processing prior to determining said level and/or chemical composition,
wherein said processing
(a) comprises fragmenting said one or both GAGs into disaccharide units; and
(b) does not comprise prior to (a) at least one of:
(i) contacting said sample with a proteolytic agent; and
(ii) purifying said one or both GAGs in said sample based on the negative charge of said GAGs.

13. The method of claim 12, wherein said method does not comprise the contacting of (b)(i) or the purifying of (b)(ii).

14. The method of claim 12 or claim 13, wherein said fragmenting of (a) is performed by contacting said one or both GAGs with one or more GAG lyase enzymes.

15. The method of claim 14, wherein said one or more GAG lyase enzymes are one or more chondroitinase enzymes and/or one or more heparinase enzymes.

16. The method of any one of claims 12 to 15, wherein said purifying of (b)(ii) is performed using an anion-exchange resin.

17. The method of any one of claims 1 to 16, wherein said level or chemical composition of said GAG property is determined by HPLC and mass spectrometry.

18. The method of claim 17, wherein said HPLC is ultra-HPLC and/or wherein said mass spectrometry is triple-quadrupole mass spectrometry.

19. The method of any one of claims 1 to 18, wherein said subject is a subject having arthritis.

20. The method of any one of claims 1 to 19, wherein said subject is a human.

21. A method of monitoring the progression of osteoarthritis (OA) or rheumatoid arthritis (RA) in a subject having arthritis, said method comprising:
determining the level and/or chemical composition of one or both of the glycosaminoglycans (GAGs) chondroitin sulfate (CS) and heparan sulfate (HS) in a urine sample,
wherein said determining the level and/or chemical composition of one or both of said GAGs comprises determining the level of one or more GAG property selected from the group consisting of:
(i) 4S CS,
(ii) NS HS,
(iii) 0S HS,
(iv) total concentration of CS; and
(v) total concentration of HS,
wherein said sample has been obtained from said subject.

22. The method of claim 21, wherein
(a) progression of OA is monitored and an altered level of said one or more GAG property over time as compared to a control level of the same one or more GAG property is indicative of progression of OA, wherein said control level has been determined in the same sample type obtained from one or more subjects not having OA or has been determined in the same sample type obtained from an earlier sample taken from the subject whose OA progression is being monitored; or
(b) progression of RA is monitored and an altered level of said one or more GAG property over time as compared to a control level of the same one or more GAG property is indicative of progression of RA, wherein said control level has been determined in the same sample type obtained from one or more subjects not having RA or has been determined in the same sample type obtained from an earlier sample taken from the subject whose RA progression is being monitored.

23. The method of claim 21 or claim 22, wherein said method has features of any one of claims 4 to 20.
